(19) United States
(12) Patent Application Publication
(10) Pub. No.: US 2025/0304657 A1
(43) Pub. Date: Oct. 2, 2025

(54) ANTIBODIES WITH NOVEL FC MODIFICATION COMBINATIONS THAT INCREASE ANTIBODY FUNCTION

(71) Applicant: Fred Hutchison Cancer Center, Seattle, WA (US)

(72) Inventors: Jim Boonyaratanakornkit, Seattle, WA (US); Matthew D. Gray, Seattle, WA (US); Justin J. Taylor, Seattle, WA (US)

(73) Assignee: Fred Hutchison Cancer Center, Seattle, WA (US)

(21) Appl. No.: 19/108,083

(22) PCT Filed: Aug. 29, 2023
(86) PCT No.: PCT/US2023/073103

§ 371 Date: Feb. 28, 2025

Related U.S. Application Data
(60) Provisional application No. 63/373,768, filed on Aug. 29, 2022.

(51) Int. Cl. C07K 16/08; A61K 39/00; A61K 47/02; A61K 47/26

(52) U.S. Cl. CPC C07K 16/08; A61K 47/02; A61K 47/26; A61K 2039/505; A61K 2039/54; C07K 2317/41; C07K 2317/52; C07K 2317/565; C07K 2317/94

(57) ABSTRACT

Antibodies with novel Fc modification combinations that increase antibody function are described. The novel Fc modification combinations include M252Y/S254T/T256E (YTE) in combination with M428L/N434S (LS) (YTE+LS), YTE+LS in combination with S239D/A330L/I332E (OLE) (YTE+LS+DLE), YTE+LS in combination with G236A/A330L/I332E (YTE+LS+ALE), YTE+LS in combination with G236A/S239D/A330L (DAL) (YTE+LS+DAL) and YTE+LS in combination with G236A/S239D/A330L/I332E (DALE) (YTE+LS+DALE). Glycosylation modifications are optionally included with these combinations.

### CROSS REFERENCE TO RELATED APPLICATION

This application is a U.S. National Phase Patent Application based on International Patent Application No. PCT/US2023/073103, filed on Aug. 29, 2023, which claims priority to U.S. Provisional Patent Application No. 63/373,768 filed Aug. 29, 2022, the entire contents of both of which are incorporated by reference herein.

### REFERENCE TO SEQUENCE LISTING

The Sequence Listing associated with this application is provided in xml format in lieu of a paper copy, and is hereby incorporated by reference into the specification. The name of the text file containing the Sequence Listing is 3DR8015.XML. The text is 106, 199 bytes, was created on Feb. 14, 2025, and is being submitted electronically via Patent Center.

### FIELD OF THE DISCLOSURE

The current disclosure provides antibodies with novel Fc modification combinations that increase antibody function and new uses of existing Fc modification combinations. The novel Fc modification combinations include the M252Y/S254T/T256E modification set (YTE) in combination with the M428L/N434S modification pair (LS) (YTE+LS), YTE+LS in combination with the S239D/A330L/I332E modification set DLE (YTE+LS+DLE), YTE+LS in combination with the G236A/A330L/I332E modification set (YTE+LS+ALE), YTE+LS in combination with the G236A/S239D/A330L modification set (YTE+LS+DAL), and YTE+LS in combination with the G236A/S239D/A330L/I332E (YTE+LS+DALE) modification set. New uses of the DAL modification set are also provided. Glycosylation modifications are optionally included with these combinations.

### BACKGROUND OF THE DISCLOSURE

Respiratory viral infections cause significant mortality, morbidity, and health care costs in hematopoietic stem cell transplant (HCT) patients. Up to 40% of patients with lower tract disease die within three months. Of patients who survive, over 25% develop air flow obstruction, a chronic debilitating condition associated with increased mortality. Collectively, two related viruses—respiratory syncytial virus (RSV) and human metapneumovirus (HMPV)—account for over a third of serious respiratory viral infections after HCT. HCT recipients are most vulnerable during the post-transplant period when their immune system has not yet fully reconstituted and they continue immunosuppressive medications to prevent graft-versus-host disease. Immune reconstitution can take months to years, and there are currently no preventative or treatment options for RSV or HMPV in HCT recipients. Passive immunization with monoclonal antibodies (mAbs) represents a strategy for protecting immunocompromised patients. A highly potent cross-neutralizing mAb against RSV and HMPV, named MxR, was recently isolated and characterized. When given as prophylaxis, MxR significantly reduced viral replication in the lungs of hamsters (FIG. 2).

Neutralization potency is generally considered one of the strongest correlates of protection against respiratory viral infections. However, other important features of mAbs can significantly influence efficacy, including Fc effector functions and pharmacokinetic (PK) properties. Fc effector functions help clear virus and virus-infected cells by activating antibody dependent cellular cytotoxicity (ADCC). In particular, Fcγ receptor IIIa (FcγRIIIa) engagement mediates protection against RSV and other respiratory viruses, such as SARS-CoV-2, in part by stimulating natural killer (NK)_cells. Fc modifications that enhance FcγRIIIa binding-like S239D/A330L/I332E DLE—can improve the potency of mAbs, such that a lower concentration is needed to suppress viral replication and protect against disease. Fcγ receptor IIa (FcγRIIa) engagement mediates protection against flu, in part by stimulating macrophages. Fcγ receptor IIb (FcγRIIb) engagement can suppress protection against viruses as this binding to this receptor inhibits immune activation. Thus, enhanced binding of an Fc to FcγRIIIa, FcγRIIa, or FcγRIIb can influence efficacy of the immune system overall as well as particular efficacy against virus types.

The efficacy of mAbs against viruses can also be improved by optimizing pharmacokinetic (PK) properties. For example, the half-life of IgG is three weeks due to intracellular uptake and degradation in lysosomes. However, IgG can also be recycled back into circulation by binding to the neonatal Fc receptor (FcRn) at an acidic pH in endosomes and dissociating from FcRn at a physiologic pH in blood or tissue. Modifications in the Fc region like M252Y/S254T/T256E (YTE) or M428L/N434S (LS) can selectively strengthen binding to FcRn at an acidic pH and prolong the half-life of IgG by up to four-fold. Interactions with FcRn also play a role in the transcytosis of IgG into the lungs, and the YTE and LS modifications also individually increase lung bioavailability by up to four-fold. Half-life extension and increased lung bioavailability are desirable properties for mAbs targeting respiratory viruses in HCT recipients who may be vulnerable to infection for several months.

While YTE and LS Fc modifications individually provide beneficial effects, the results of their combination could not be reasonably predicted. For example, in designing antibodies with increased half-life, it is generally beneficial to have enhanced target binding at the acidic pH of lysosomes, but not at the physiological pH of the bloodstream. Combining the increased binding of YTE and LS could result in too strong of an interaction, sequestering the antibody at the cell surface, rather than allowing its release into the systemic circulation. Accordingly, there are many factors and potential outcomes that can affect the impact of Fc modifications, and these outcomes are unpredictable.

DE, DLE, ALE, and DALE Fc modification sets are individually known to increase binding to FcγRIIIa. DE, ALE, and DALE Fc modification sets additionally individually increase binding to FcγRIIa, while the DLE modification set decreases binding to FcγRIIa. Unfortunately, each of these modifications similarly increases binding to the immune inhibitory FcγRIIb receptor.

### SUMMARY OF THE DISCLOSURE

The current disclosure provides novel combinations of Fc modifications that unexpectedly significantly increase binding to FcRn. These Fc modifications include the M252Y/S254T/T256E (YTE) modification set in combination with the M428L/N434S (LS) modification pair (YTE+LS). Additional combinations that significantly increase binding to FcRn include YTE+LS in combination with the S239D/A330L/I332E DLE modification set (YTE+LS+DLE), YTE+LS in combination with the G236A/A330L/I332E modification set (YTE+LS+ALE), and YTE+LS in combination with the G236A/S239D/A330L/I332E modification set (YTE+LS+DALE). These novel combinations can augment the 1) half-life; 2) lung bioavailability; and 3) potency of neutralizing mAb in vivo.

Fc modifications also impact binding to FcγRIIIa, FcγRIIa, and FcγRIIb, affecting immune activating and suppressing properties. As disclosed herein, the YTE+LS+DLE, YTE+LS in combination with the G236A/S239D/A330L modification set (YTE+LS+DAL), YTE+LS+DALE modification sets and YTE+LS with glycosylation modifications (YTE+LS+M5) individually significantly increase binding to FcγRIIIa with no increased binding to FcγRIIb. This profile provides particular suitability for use of these Fc modification set combinations for treating respiratory viruses, such as human parainfluenza viruses (HPIV), respiratory syncytial virus (RSV), human metapneumovirus (HMPV), and SARS-CoV-2. The YTE+LS+DAL modification set also significantly increases binding to FcγRIIa, suggesting that this modification set can provide more protection against a broader range of viruses, including flu. The YTE+LS+ALE modification set can be particularly effective in treating a wide range of viruses (e.g., respiratory viruses and flu) because this modification set has increased binding to FcγRIIIa and FcγRIIa with decreased binding to the immune-suppressing FcγRIIb. The DAL modification set also provides a modification set that can be particularly effective in the treatment of flu because it significantly increases binding to FcγRIIa but is not different from wild-type IgG antibodies in terms of binding to FcγRIIa and FcγRIIb.

As described herein, the YTE+LS modification set is particularly well-suited for prophylactic antibody treatments. Antibodies with the YTE+LS modification set half long circulating half-lives and are particularly appropriate for intramuscular (IM) or intravenous (IV) administration.

As described herein, the ALE, DALE, and YTE+LS+DAL modification sets and glycosylation modifications are individually particularly well-suited for therapeutic treatments through inhalation.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. Exemplary use of antibodies disclosed herein to bridge windows of vulnerability in hematopoietic stem cell transplant recipients.

FIG. 2. Efficacy of prophylactic administration of cross-neutralizing mAb MxR in vivo. Hamsters were injected intramuscularly with 5 mg/kg of MxR two days prior to intranasal challenge with 10⁵ pfu of virus. RSV (a) and HMPV (b) titers by plaque assay in lungs at day five post-infection. N=8-10, pooled from two independent experiments. Dashed lines indicate the limit of detection. Bars represent the mean, and asterisks indicate P<0.01 by Mann-Whitney test compared to control hamsters injected with PBS.

FIG. 3. Binding kinetics of Fc variants to FcRn. Biolayer interferometry analysis of binding between human FcRn and wild-type (WT) M×R, M×R^{YTE}, M×R^{LS}, and M×R^{YTE+LS} at (a) pH 6 and (b) pH 7.4. (c) Summary data of binding affinity (K_{D}), association constant (Kₒₙ), and dissociation constant (K_{off}).

FIG. 4. Binding kinetics of Fc variants to human FcγRIIIa. Biolayer interferometry analysis of wild-type, YTE-, and YTE+LS+DLE-modified MxR binding to low affinity human FcγRIIIa.

FIGS. 5A, 5B. Binding kinetics. (5A) Table of binding kinetics by biolayer interferometry of Fc variants to FcRn at pH 6 and FcγRIIIa. For FcRn, the concentration of FcRn was 0.5 M and the concentrations of antibodies were 267, 133, 68, and 33 nM. For FcγRIIIa, the concentration of FcγRIIIa was 0.25 UM and the concentrations of antibodies were 1667, 834, 417, and 208 nM. (5B) Binding kinetics by biolayer interferometry of FcRn at pH 6 to Fc variants at 267 nM.

FIGS. 6A-6C. Binding kinetics of Fc variants to human FcγRIIIa (6A), FcγRIIa (6B), and FcγRIIb (6C) for Fc variants WT, DAL, DALE, DE, DLE, and ALE.

FIGS. 7A-7C. Binding kinetics of Fc variants to human FcγRIIIa (7A), FcγRIIa (7B), and FcγRIIb (7C) for Fc variants WT, YTE+LS+DAL, YTE+LS+DALE, YTE+LS+DLE, and YTE+LS+ALE.

FIGS. 8A-8D. (8A) Humanized transgenic FcRn mice were injected intravenously with 5 mg/kg M×R containing no modifications in the Fc region (wild-type or WT) or YTE, LS, or YTE+LS Fc modifications. Serum was collected on day 1, 7, 14, and 28 post-injection. M×R concentration was measured by ELISA. (8B) A two-phase exponential decay model was used to calculate the slow-phase half-life of MxR Fc variants. (8C, 8D) Humanized transgenic FcRn mice were injected intravenously with 5 mg/kg MxR containing YTE, LS, or YTE+LS Fc modifications. Bronchoalveolar lavage (BAL) was performed and serum was collected on day 28. MxR concentration was measured by ELISA and normalized to urea concentrations in the BAL and serum.

FIGS. 9A, 9B. (9A) Transgenic humanized Fc gamma receptor mice were injected intravenously with 5 mg/kg MxR with no Fc modifications (wild-type or WT) or with the DLE Fc modification. Serum was collected on days 1, 7, 14, and 28 post-injection. MxR concentration was measured by ELISA. (9B) Transgenic humanized FcRn mice were injected intravenously with 5 mg/kg MxR with no Fc modifications (wild-type or WT) or with the DLE or YTE+LS+DLE Fc modification. Serum was collected on days 1, 14, and 28 post-injection. MxR concentration was measured by ELISA.

FIG. 10. Fc glycosylation modifications increases binding to FcγRIIIa without affecting FcγRIIa or FcγRIIb binding. Monoclonal antibodies (mAbs) were produced in GnTI-293 cells to have an Fc region glycosylated with five mannoses and no fucose.

FIG. 11. Efficacy of inhaled Fc modified mAbs in hamsters. Hamsters were infected with 100,000 pfu HMPV. M×R^{WT} or M×R^{ALE} were administered intranasally at 2.5 mg/kg on day 1 post-infection. Nasal turbinates were harvested on day 4 post-infection. A significant reduction in viral replication in hamsters treated with M×R^{ALE} was observed. A trend towards reduction in viral replication with M×R^{WT} was also observed but was not statistically significant.

FIG. 12. Summary Table of Fc modification set combination effects and exemplary clinical uses.

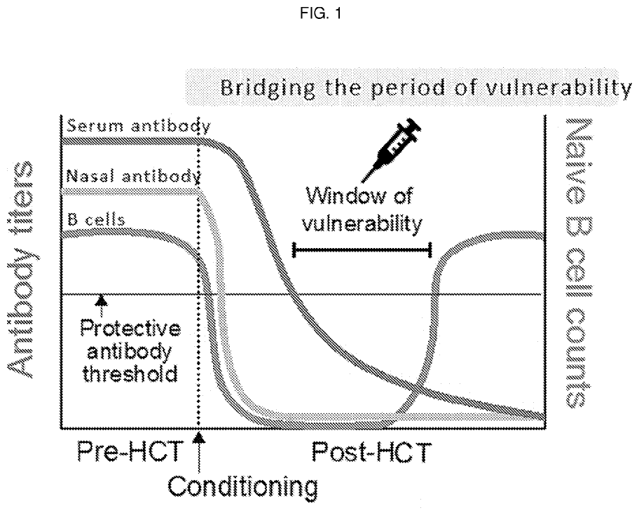

### DETAILED DESCRIPTION

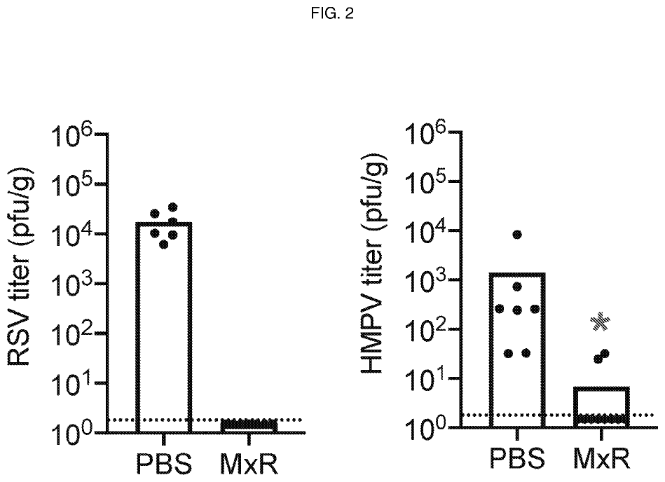

Respiratory viral infections cause significant mortality, morbidity, and health care costs in hematopoietic stem cell transplant (HCT) patients. Up to 40% of patients with lower tract disease die within three months. Of patients who survive, over 25% develop air flow obstruction, a chronic debilitating condition associated with increased mortality. Erard et al., J Infect Dis 193, 1619-1625 (2006); Chien et al., Am J Respir Crit Care Med 168, 208-214, (2003). Collectively, two related viruses—RSV and HMPV—account for over a third of serious respiratory viral infections after HCT. Erard et al., J Infect Dis 193, 1619-1625 (2006); Boeckh et al., Br J Haematol 143, 455-467, (2008). HCT recipients are most vulnerable during the post-transplant period when their immune system has not yet fully reconstituted and they continue immunosuppressive medications to prevent graft-versus-host disease. Boyarsky et al., JAMA, doi: 10.1001/jama.2021.4385 (2021) PMC7961463; Agha et al., medRxiv, 2021.2004.2006.21254949, doi: 10.1101/2021.04.06.21254949 (2021). Immune reconstitution can take months to years, and there are currently no preventative or treatment options for RSV or HMPV in HCT recipients. Eberhardt et al., Curr Opin Infect Dis 34, 275-287, (2021); Yanir et al., Front Pediatr 9, 786017, (2021). Passive immunization with monoclonal antibodies (mAbs) represents a strategy for protecting immunocompromised patients. A highly potent cross-neutralizing mAb against RSV and HMPV, named MxR, was recently isolated and characterized. When given as prophylaxis, MxR significantly reduced viral replication in the lungs of hamsters (FIG. 2).

Neutralization potency is generally considered one of the strongest correlates of protection against respiratory viral infections. Zohar et al., Cell Host Microbe 30, 41-52 e45, (2022); Khoury et al., Nat Med 27, 1205-1211, (2021); Gilbert et al., Science 375, 43-50, (2022); Maas et al., EBioMedicine 73, 103651, (2021). However, other important features of mAbs can significantly influence efficacy, including Fc effector functions and pharmacokinetic (PK) properties. Fc effector functions help clear virus and virus-infected cells by activating antibody dependent cellular cytotoxicity (ADCC). In particular, Fcγ receptor IIIa (FcγRIIIa) engagement mediates protection against RSV and other respiratory viruses. Zohar et al., Cell Host Microbe 30, 41-52 e45, (2022); Yamin et al., Nature 599, 465-470, (2021). Fc modifications that enhance FcγRIIIa binding—like S239D/A330L/I332E (DLE)—can improve the potency of mAbs, such that a lower concentration is needed to suppress viral replication and protect against disease. Yamin et al., Nature 599, 465-470, (2021).

The efficacy of mAbs against respiratory viruses can also be improved by optimizing PK properties. The half-life of IgG is three weeks due to intracellular uptake and degradation in lysosomes. Ryman et al., CPT Pharmacometrics Syst Pharmacol 6, 576-588, (2017); Saunders et al., Front Immunol 10, 1296, (2019). However, IgG can also be recycled back into circulation by binding to the neonatal Fc receptor (FcRn) at an acidic pH in endosomes and dissociating from FcRn at a physiologic pH in blood or tissue. Modifications in the Fc region like M252Y/S254T/T256E (YTE) or M428L/N434S (LS) can selectively strengthen binding to FcRn at an acidic pH and prolong the half-life of IgG by up to four-fold. Spiekermann et al., J Exp Med 196, 303-310, (2002); Heidl et al., Protoplasma 253, 1557-1564, (2016); Dall'Acqua et al., J Biol Chem 281, 23514-23524, (2006); Ko et al., Nature 514, 642-645, (2014). Interactions with FcRn also play a role in the transcytosis of IgG into the lungs (Ryman et al., CPT Pharmacometrics Syst Pharmacol 6, 576-588, (2017)), and the YTE and LS modifications also individually increase lung bioavailability by up to four-fold. Dall'Acqua et al., J Biol Chem 281, 23514-23524, (2006). Half-life extension and increased lung bioavailability are desirable properties for mAbs targeting respiratory viruses in HCT recipients who may be vulnerable to infection for several months.

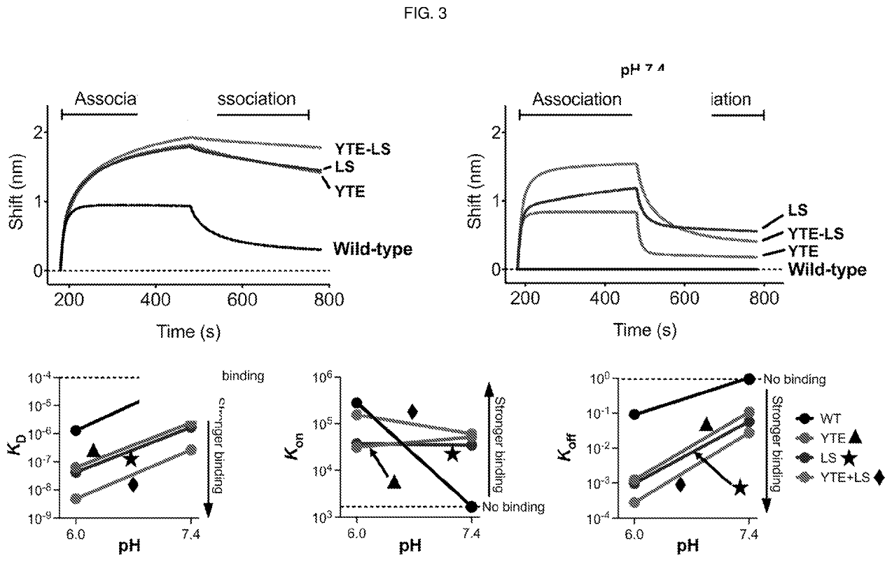

The individual Fc modifications YTE and LS were first reported in 2002 and 2010, respectively. Acqua et al., J. Immunol. 169 (9) 5171-5180 (2002); Zalevsky et al., Nature Biotechnology 28, 157-159, 2010. Before the current disclosure, it was not known whether the PK profile of mAbs could be further optimized beyond the four-fold improvement conferred by YTE or LS individually because the YTE and LS modifications had not been combined experimentally. It was also not known whether combining YTE+LS would have an adverse, zero, additive, or synergistic effect on FcRn binding. As disclosed herein, four different Fc variants of M×R (M×R, MxR^{YTE}, M×R^{LS}, M×R^{YTE+LS}) were produced and their affinity to human FcRn (FIG. 3) was measured. At pH 6, the doubly modified M×R^{YTE+LS} had over 270-fold stronger binding affinity (KD) to FcRn compared to wild-type MxR and almost 10-fold greater binding affinity compared to the single mutants M×R^{YTE} and M×R^{LS}. At pH 7.4, M×R^{YTE+LS} had a dissociation rate (K_{off}) comparable to M×R^{LS}. Together, these data show that M×R^{YTE+LS} can have a longer half-life and greater respiratory bioavailability than M×R^{YTE} and M×R^{LS}.

While YTE and LS Fc modifications individually provide beneficial effects, the results of their combination could not be reasonably predicted. For example, in designing antibodies with increased half-life, it is generally beneficial to have enhanced target binding at the acidic pH of lysosomes, but not at the physiological pH of the bloodstream. Combining the increased binding of YTE and LS could result in too strong of an interaction, sequestering the antibody at the cell surface, rather than allowing its release into the systemic circulation. Accordingly, there are many factors and potential outcomes that can affect the impact of Fc modifications, and these outcomes are unpredictable.

DE, DLE, ALE, and DALE Fc modification sets are individually known to increase binding to FcγRIIIa. DE, ALE, and DALE Fc modification sets additionally individually increase binding to FcγRIIa, while the DLE modification set decreases binding to FcγRIIa. Unfortunately, each of these modifications similarly increases binding to the immune inhibitory FcγRIIb receptor.

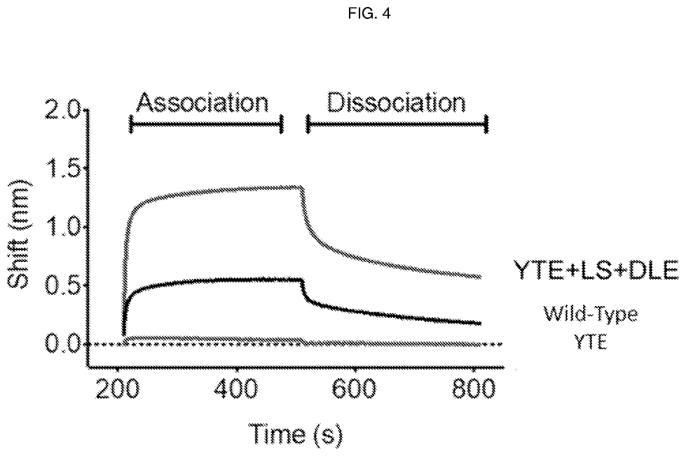

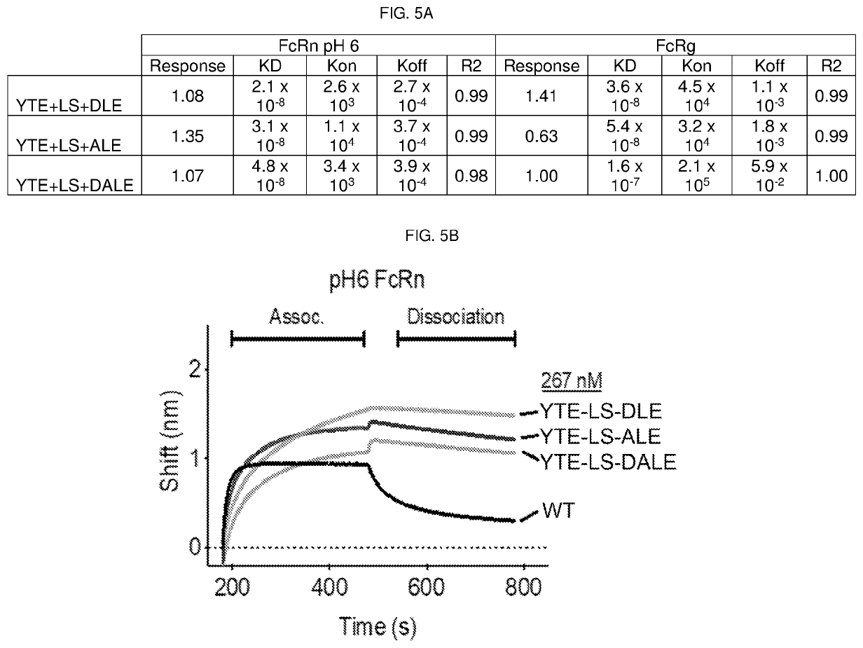

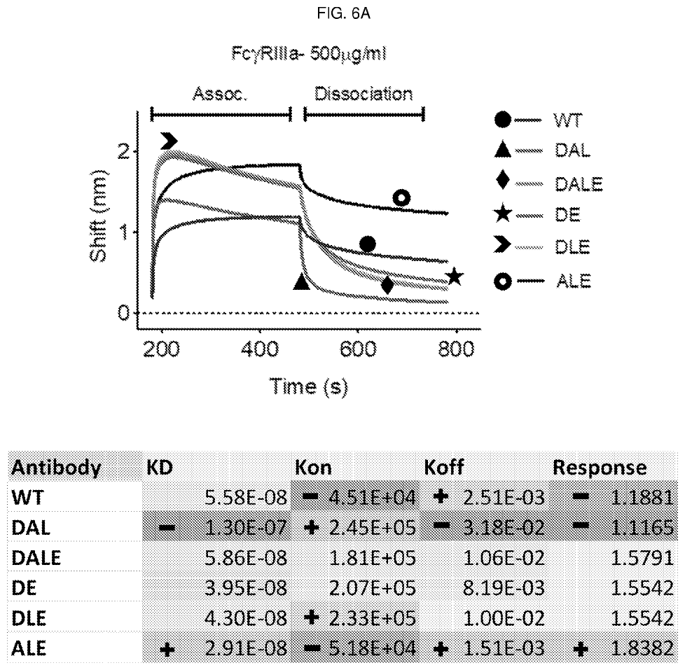

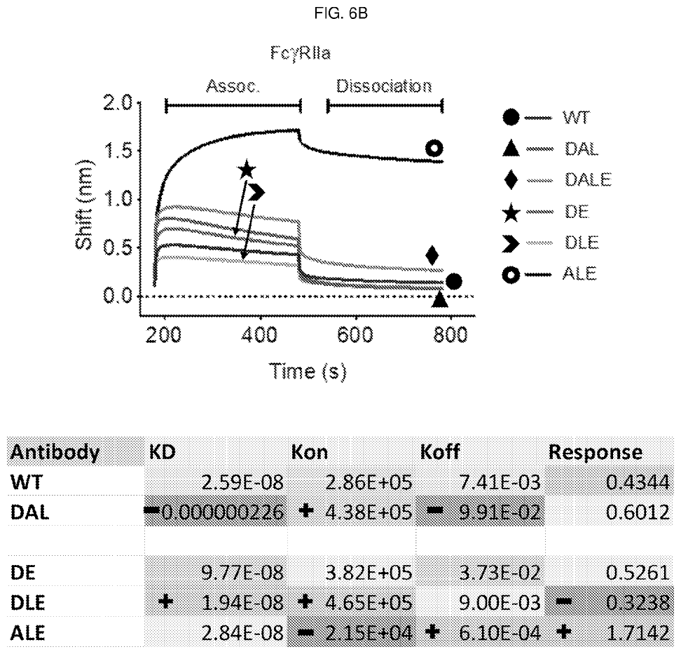

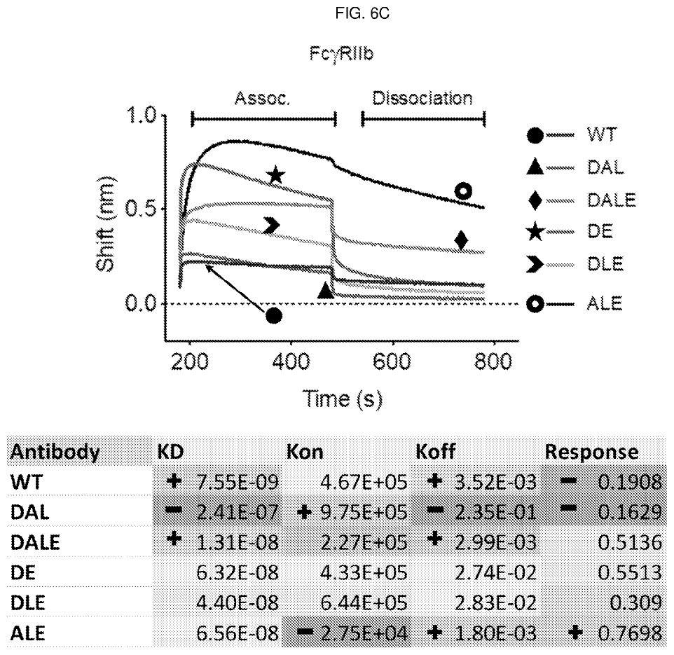

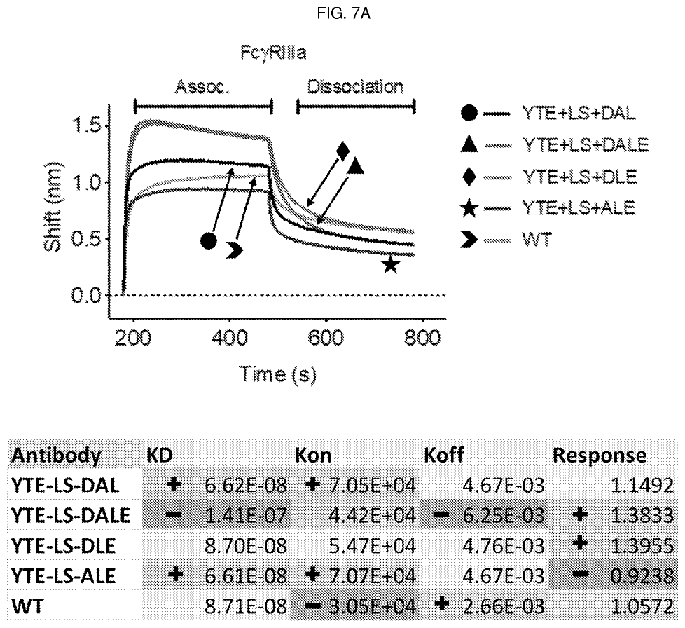

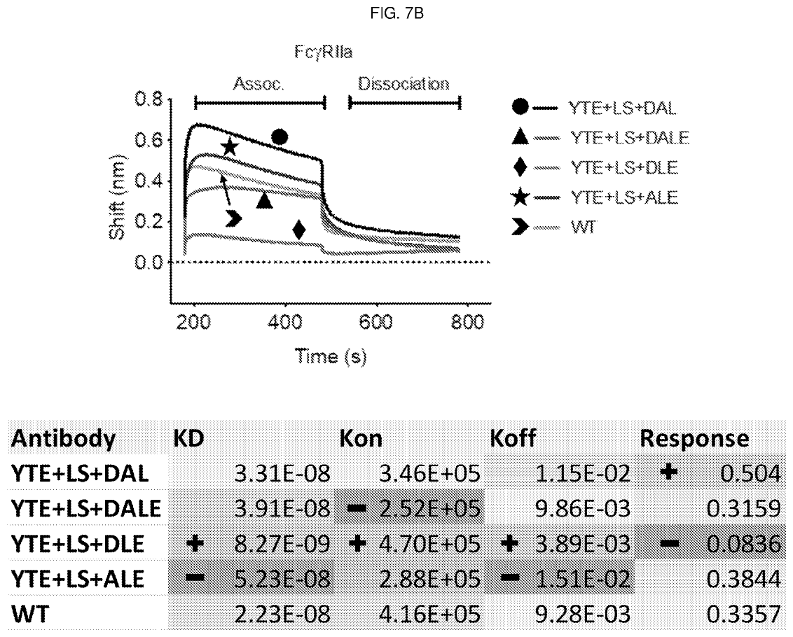

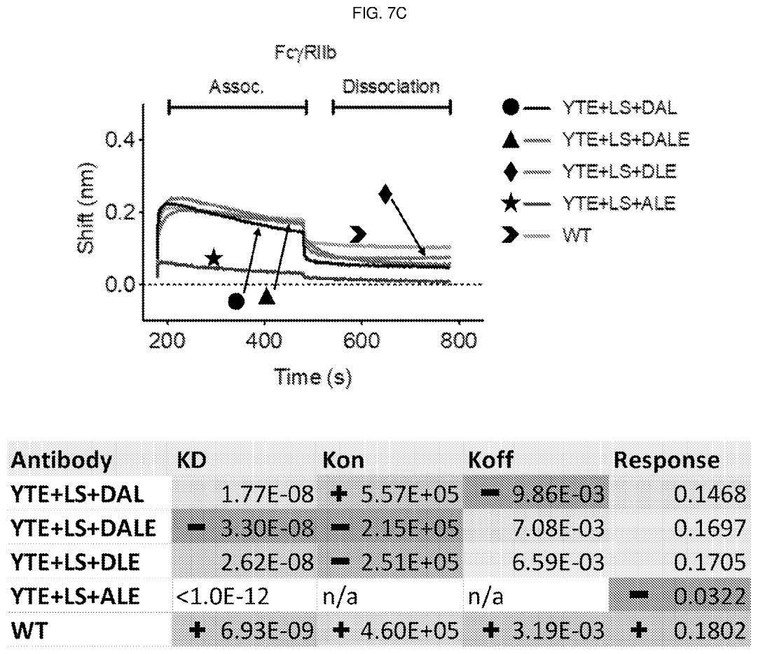

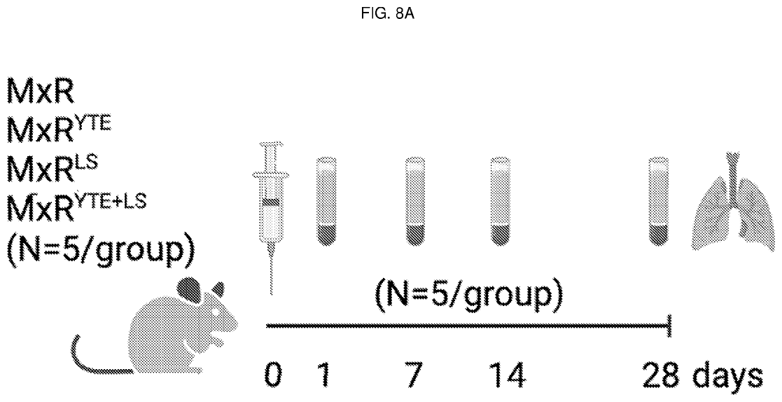

While the YTE modification increases FcRn binding affinity, it significantly reduces Fγg receptor engagement. The DLE modification set can counteract this phenomenon and boost FγgRIIIa binding and ADCC. (Dall'Acqua et al., J Biol Chem 281, 23514-23524, (2006)). The binding affinities of M×R, M×R^{YTE}, and M×R^{YTE+LS+DLE} with human FγgRIIIa were compared. As expected, M×R^{YTE} did not bind, wild-type M×R bound weakly, and M×R^{YTE+LS+DLE} bound strongly to human FcγRIIIa (FIG. 4).

Thus, the current disclosure provides novel combinations of Fc modifications that unexpectedly significantly increase binding to FcRn. These Fc modifications include the M252Y/S254T/T256E (YTE) modification set in combination with the M428L/N434S (LS) modification pair (YTE+LS). Additional combinations that significantly increase binding to FcRn include YTE+LS in combination with the S239D/A330L/I332E DLE modification set (YTE+LS+DLE), YTE+LS in combination with the G236A/A330L/I332E modification set (YTE+LS+ALE), and YTE+LS in combination with the G236A/S239D/A330L/I332E modification set (YTE+LS+DALE). These novel combinations can augment the 1) half-life; 2) lung bioavailability; and 3) potency of neutralizing mAb in vivo.

As disclosed herein, the YTE+LS+DLE, YTE+LS in combination with the G236A/S239D/A330L modification set (YTE+LS+DAL), and YTE+LS+DALE modification sets and YTE+LS with glycosylation modifications (YTE+LS+M5) individually significantly increase binding to FcγRIIIa with no increased binding to FcγRIIb. This profile provides particular suitability for use of these Fc modification combinations for treating respiratory viruses, such as human parainfluenza viruses (HPIV), respiratory syncytial virus (RSV), human metapneumovirus (HMPV), and SARS-CoV-2. YTE+LS+DAL also significantly increases binding to FcγRIIa, suggesting that this modification set can provide more protection against a broader range of viruses, including flu. The YTE+LS+ALE modification set can be particularly effective in treating a wide range of viruses (e.g., respiratory viruses and flu) because this modification set has increased binding to FcγRIIIa and FcγRIIa with decreased binding to the immune-suppressing FcγRIIb. The DAL modification set also provides a modification set that can be particularly effective in the treatment of flu because it significantly increases binding to FcγRIIa but is not different from wild-type IgG antibodies in terms of binding to FcγRIIa and FcγRIIb.

As described herein, the YTE+LS modification set is particularly well-suited for prophylactic antibody treatments. Antibodies with the YTE+LS modification set half long circulating half-lives and are particularly appropriate for intramuscular (IM) or intravenous (IV) administration.

As described herein, the ALE, DALE, and YTE+LS+DAL modification sets as well as glycosylation modifications (e.g., M5 or YTE+LS+M5) are individually particularly well-suited for therapeutic treatments through inhalation.

Particular embodiments disclosed herein exclude modifications beyond those combinations specifically recited. Particular embodiments exclude modification N434H. Particular embodiments exclude modification C220S. Particular embodiments exclude modifications N434H and C220S.

Aspects of the current disclosure are now described in more supporting detail as follows: (i) Antibodies and Fc Modifications; (ii) Compositions; (iii) Methods of Use; (iv) Exemplary Embodiments; (v) Prophetic Examples; and (vi) Closing Paragraphs. These headings are provided for organizational purposes only and do not limit the scope or interpretation of the disclosure.

(i) Antibodies and Fc Modifications. Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme)), AHo (Honegger A and Pluckthun A, “Yet another numbering scheme for immunoglobulin variable domains: an automatic modeling and analysis tool,” J Mol Biol, 2001 Jun. 8; 309 (3): 657-70, (AHo numbering scheme)), North (North et al., J Mol Biol. 406 (2): 228-256 (2011), “A new clustering of antibody CDR loop conformations”), or other numbering schemes.

Software programs and bioinformatical tools, such as ABodyBuilder and Paratome can also be used to determine CDR sequences. Additionally, delineation of a CDR can be according to X-ray crystallography.

The carboxy-terminal portion of each chain defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g. B-cell receptors); and B-cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

As indicated, antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

In particular embodiments, an epitope denotes the binding site on a viral peptide bound by a corresponding variable region of an antibody. The variable region either binds to a linear epitope, (e.g., an epitope including a stretch of 5 to 12 consecutive amino acids), or the variable region binds to a three-dimensional structure formed by the spatial arrangement of several short stretches of the protein target. Three-dimensional epitopes recognized by a variable region, e.g. by the epitope recognition site or paratope of an antibody or antibody fragment, can be thought of as three-dimensional surface features of an epitope molecule. These features fit precisely (in) to the corresponding binding site of the variable region and thereby binding between the variable region and its target protein (more generally, antigen) is facilitated. In particular embodiments, an epitope can be considered to have two levels: (i) the “covered patch” which can be thought of as the shadow an antibody variable region would cast on the antigen to which it binds; and (ii) the individual participating side chains and backbone residues that facilitate binding. Binding is then due to the aggregate of ionic interactions, hydrogen bonds, and hydrophobic interactions.

Epitopes of the currently disclosed antibodies (that is, epitopes to which the antibodies bind) are found on a virus selected from HPIV3, HPIV1, RSV, and/or HMPV. In particular embodiments, the epitope is located within a viral F protein, for example in its prefusion state.

In particular embodiments, “bind” means that the variable region associates with its target epitope with a dissociation constant (Kd or KD) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷M, in particular embodiments of from 10⁻⁸ M to 10⁻¹³ M, or in particular embodiments of from 10⁻⁹ M to 10⁻¹³ M. The term can be further used to indicate that the variable region does not bind to other biomolecules present (e.g., it binds to other biomolecules with a dissociation constant (Kd) of 10⁻⁴ M or more, in particular embodiments of from 10⁻⁴ M to 1 M).

In particular embodiments, Kd can be characterized using BIAcore. For example, in particular embodiments, Kd can be measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) can be activated with N-ethyl-N′-(3-dimethylaminopropyl)-carbodiimide hydrochloride (EDC) and N-hydroxysuccinimide (NHS) according to the supplier's instructions. Antigen can be diluted with 10 mM sodium acetate, pH 4.8, to 5 μg/ml (0.2 μM) before injection at a flow rate of 5 μl/minute to achieve 10 response units (RU) of coupled protein. Following the injection of antigen, 1 M ethanolamine can be injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of 25 μl/min. Association rates (Kₒₙ) and dissociation rates (K_{off}) can be calculated using a simple one-to-one Langmuir binding model (BIACORE® Evaluation Software version 3.2) by simultaneously fitting the association and dissociation sensorgrams. The equilibrium dissociation constant (Kd) can be calculated as the ratio K_{off}/Kₒₙ. See, e.g., Chen et al., J. Mol. Biol. 293:865-881, 1999. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Unless otherwise indicated, the term “antibody” includes (in addition to antibodies having two full-length heavy chains and two full-length light chains as described above) includes variants, derivatives, and fragments thereof, examples of which are described below, so long as the antibody includes a mutated Fc region as disclosed herein. Furthermore, unless explicitly excluded, antibodies can include monoclonal antibodies, human or humanized antibodies, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring modifications or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup III as in Kabat et al. (supra).

A “humanized” antibody refers to a chimeric antibody including amino acid residues from non-human CDRs and amino acid residues from human FRs. In particular embodiments, a humanized antibody will include substantially all of at least one, and typically two, variable domains, in which all or substantially all of the CDRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

Humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498, 1991 (describing “resurfacing”); Dall'Acqua et al., Methods 36:43-60,2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Nati. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-22618, 1996).

In particular embodiments, mAb PI3-E12 has a CDRH1 including GFTFSDHY (SEQ ID NO: 1); a CDRH2 including ISSSGSNT (SEQ ID NO: 2); a CDRH3 including ARAKWGTMGRGAPPTIYDH (SEQ ID NO: 3); a CDRL1 including QSLLQSNGNNY (SEQ ID NO: 4); a CDRL2 including LGS; and a CDRL3 including MQALQTPLT (SEQ ID NO: 5).

In particular embodiments, PI3-E12 has a heavy chain sequence including QVQLLESGGKLVKPGGSLRLSCAASGFTFSDHYMIWIRQAPGKGLEWISYISSSGSNTIYADSL MGRFTISRDNAKNSLYLQMNSLRTEDTAVYYCARAKWGTMGRGAPPTIYDHWGQGTLVTVSS (SEQ ID NO: 166) and a light chain sequence including DIVMTQSPLSLPVTPGEPASISCRSSQSLLQSNGNNYLEWYLQKPGQSPQLLIYLGSNRASGVP DRFSGSGSGTDFTLKISRVEAEDAGVYYCMQALQTPLTFGGGTKVEIK (SEQ ID NO: 167).

In particular embodiments, the PI3-E12 antibody includes a variable heavy chain sequence encoded by: CAGGTGCAGCTGTTGGAGTCTGGGGGAAAGTTGGTCAAGCCTGGAGGGTCCCTGAGACT CTCCTGTGCAGCCTCTGGATTCACCTTCAGTGACCACTACATGATCTGGATCCGCCAGGCT CCCGGGAAGGGGCTGGAGTGGATTTCATACATAAGTAGTAGTGGTAGTAACACAATCTAC GCAGACTCTTTGATGGGCCGATTCACCATCTCCAGAGACAACGCCAAGAACTCTCTGTATC TACAAATGAACAGCCTGAGGACCGAGGACACGGCCGTTTATTACTGTGCGAGAGCAAAGT GGGGTACTATGGGTCGGGGAGCACCCCCGACAATTTATGACCACTGGGGCCAGGGAACC CTGGTCACCGTCTCCTCA (SEQ ID NO: 6) and a variable kappa light chain sequence encoded by:

(SEQ ID NO: 7)
GATATTGTGATGACTCAGTCTCCACTCTCCCTGCCCGTCACCCCTGGAG
AGCCGGCCTCCATCTCCTGTAGGTCTAGTCAGAGCCTCCTGCAAAGTAA
TGGAAACAATTATTTGGAGTGGTACCTGCAGAAGCCAGGGCAGTCTCCA
CAACTCCTGATCTATTTGGGTTCCAATCGGGCCTCCGGGGTCCCTGACA
GGTTCAGTGGCAGTGGATCAGGCACAGATTTTACACTGAAGATCAGCAG
AGTGGAGGCTGAGGATGCTGGGGTTTATTACTGCATGCAAGCTCTACAA
ACTCCGCTCACTTTCGGCGGAGGGACCAAGGTGGAGATCAAAC.

In particular embodiments, mAb PI3-A3 has a CDRH1 including GFTFSNYW (SEQ ID NO: 8); a CDRH2 including VKEEGSEK (SEQ ID NO: 9); a CDRH3 including AGEVKSGWFGRYFDS (SEQ ID NO: 10); a CDRL1 including QSVGSW (SEQ ID NO: 11); a CDRL2 including KTS; and a CDRL3 including QQYSSFPYT (SEQ ID NO: 12).

In particular embodiments mAb PI3-A3 has a heavy chain sequence including EVOLVESGGGLVQPGGSLRLSCTASGFTFSNYWMSWVRQAPGKGLEWVANVKEEGSEKHY VDSVKGRFTISRDNAKNSVYLQMSSLRAEDTAVYYCAGEVKSGWFGRYFDSWGQGTLVTVSS (SEQ ID NO: 168) and a light chain sequence including

(SEQ ID NO: 169)
DIQMTQSPSTLSASVGDRVTINCRASQSVGSWLAWYQQKPGKAPKLLMY
KTSTLQRGVPSRFSGSGSGTEFTLTISSLQPDDFAAYYCQQYSSFPYTF
GQGTKLEIK.

In particular embodiments, the PI3-A3 antibody includes a variable heavy chain sequence encoded by: GAGGTGCAGCTGGTGGAGTCTGGGGGAGGCTTGGTCCAGCCTGGGGGGTCCCTGAGACT CTCCTGTACAGCCTCTGGATTCACCTTTAGCAATTATTGGATGAGCTGGGTCCGCCAGGCT CCAGGGAAGGGGCTGGAGTGGGTGGCCAATGTGAAGGAAGAAGGAAGTGAGAAACACTA TGTAGACTCTGTGAAGGGCCGATTCACCATCTCCAGAGACAACGCCAAGAACTCAGTGTAT CTGCAGATGAGCAGCCTGAGAGCCGAGGACACGGCTGTCTATTACTGTGCGGGAGAGGTT AAGAGTGGCTGGTTCGGTCGGTACTTTGACTCCTGGGGCCAGGGAACCCTGGTCACCGTC TCCTCAG (SEQ ID NO: 13) and a variable kappa light chain sequence encoded by:

(SEQ ID NO: 14)
GACATCCAGATGACCCAGTCTCCTTCCACCCTGTCTGCATCTGTTGGAG
ACAGAGTCACCATCAATTGCCGGGCCAGTCAGAGTGTTGGTAGCTGGTT
GGCCTGGTATCAGCAGAAACCAGGGAAAGCCCCTAAGCTCCTGATGTAT
AAGACATCTACTTTACAAAGAGGGGTCCCATCAAGGTTCAGCGGCAGTG
GATCTGGGACAGAATTCACTCTCACCATCAGCAGCCTGCAGCCTGATGA
TTTTGCAGCTTATTACTGCCAACAGTATAGTAGTTTTCCGTACACTTTT
GGCCAGGGGACCAAGCTGGAGATCAAAC.

In particular embodiments, mAb PI3-B5 has a CDRH1 including GYNFTNYW (SEQ ID NO: 15); a CDRH2 including IYPADSDT (SEQ ID NO: 16); a CDRH3 including ARPSTRWFVPGGMDV (SEQ ID NO: 17); a CDRL1 including QSIGAW (SEQ ID NO: 18); a CDRL2 including KAS; and a CDRL3 including QQHSSYPST (SEQ ID NO: 19).

In particular embodiments, mAb PI3-B5 has a heavy chain sequence including EVQLVQSGAEVKKPGESLRISCKGSGYNFTNYWIAWVRQMPGKGLEWMGIIYPADSDTRYSP SFQGQVTISADKSITTAYLQWSSLKASDTAIYYCARPSTRWFVPGGMDVWGQGTTVIVSS (SEQ ID NO: 170) and a light chain sequence including

(SEQ ID NO: 171)
DIQMTQSPSTLSASVGDRVTISCRATQSIGAWLAWYQQKPGEPPKLLIY
KASTLESGVPSRFSGSGSGTEFTLTISSLQPDDSATYYCQQHSSYPSTF
GGGTKVEIK.

In particular embodiments, the PI3-B5 antibody includes a variable heavy chain sequence encoded by: GAGGTGCAGCTGGTGCAGTCTGGAGCAGAGGTGAAAAAGCCCGGGGAGTCTCTGAGGAT CTCCTGTAAGGGTTCTGGATACAACTTTACCAACTACTGGATCGCCTGGGTGCGCCAGATG CCCGGGAAAGGCCTGGAGTGGATGGGGATCATCTATCCTGCTGACTCGGATACCAGATAC AGCCCGTCCTTCCAAGGCCAAGTCACCATCTCAGCCGACAAGTCCATCACCACCGCCTAC CTGCAGTGGAGCAGCCTGAAGGCCTCGGACACCGCCATATACTACTGTGCGAGACCGAGT ACTAGGTGGTTCGTCCCTGGCGGTATGGACGTCTGGGGCCAAGGCACCACGGTCATCGT CTCCTCA (SEQ ID NO: 20) and a variable kappa light chain sequence encoded by:

(SEQ ID NO: 21)
GACATCCAGATGACCCAGTCTCCTTCCACCCTGTCTGCTTCTGTAGGAG
ACAGAGTCACCATTTCTTGCCGGGCCACTCAGAGTATTGGTGCCTGGTT
GGCCTGGTATCAGCAGAAACCAGGGGAACCCCCTAAGCTCCTGATCTAT
AAGGCGTCTACTTTAGAGAGTGGGGTCCCATCAAGGTTCAGCGGCAGTG
GATCTGGGACAGAATTCACTCTCACCATCAGCAGCCTGCAGCCTGATGA
TTCTGCAACTTATTACTGCCAACAGCATAGTAGTTATCCTTCTACTTTC
GGCGGAGGGACCAAGGTGGAGATCAAAC.

In particular embodiments, mAb PI3-A10 has a CDRH1 including GFNFNNYG (SEQ ID NO: 22); a CDRH2 including VSFDGSNR (SEQ ID NO: 23); a CDRH3 including SKSKYSDFWSEI (SEQ ID NO: 24); a CDRL1 including QNVMRY (SEQ ID NO: 25); a CDRL2 including DAS; and a CDRL3 including QQRTNHRFS (SEQ ID NO: 26).

In particular embodiments, mAb PI3-A10 has a heavy chain sequence including QVQLVESGGGVVRPGRSLRLSCVASGFNFNNYGLQWIRQAPGKGLEWVAGVSFDGSNRYYA DSVKGRVTISRDDSKNTLYLEMNSLRAEDTGIYYCSKSKYSDFWSEIWGQGTLVTVSS (SEQ ID NO: 172) and a light chain sequence including

(SEQ ID NO: 173)
EIVLTQSPATLSLSPGERATLSCRASQNVMRYLAWYQQRPGQAPRLLFY
DASSRATGIPARFTASGSGTDFTLTISGLEPGDFAVYYCQQRTNHRFSF
GPGTKVDIK.

In particular embodiments, the PI3-A10 antibody includes a variable heavy chain sequence encoded by: CAGGTGCAGCTGGTGGAGTCTGGGGGAGGCGTGGTCCGGCCTGGGAGGTCCCTGAGAC TCTCCTGTGTAGCCTCTGGATTCAACTTCAATAACTATGGGCTGCAGTGGATCCGCCAGGC TCCAGGCAAGGGGCTGGAGTGGGTGGCAGGTGTCTCGTTTGATGGGAGTAATAGATATTA TGCAGACTCCGTGAAGGGCCGAGTCACCATATCCAGAGACGATTCCAAGAACACCCTGTA TCTAGAAATGAACAGCCTGAGAGCTGAGGACACAGGAATATATTACTGTTCGAAGTCCAAG TACTCCGACTTTTGGAGCGAAATATGGGGCCAGGGAACCCTGGTCACCGTCTCCTCAG (SEQ ID NO: 27) and a variable kappa light chain sequence encoded by:

(SEQ ID NO: 28)
GAAATTGTGTTGACACAGTCTCCAGCCACCCTGTCTTTGTCTCCAGGGG
AAAGAGCCACCCTCTCCTGCAGGGCCAGTCAGAATGTTATGAGGTACTT
AGCCTGGTACCAACAGAGACCTGGCCAGGCTCCCAGACTCCTCTTCTAT
GATGCATCCAGCCGGGCCACTGGCATCCCAGCCCGGTTCACTGCCAGTG
GGTCTGGGACAGACTTCACTCTCACCATCAGCGGCCTCGAGCCTGGAGA
TTTTGCAGTTTATTACTGTCAGCAGCGTACCAACCATAGATTCTCTTTC
GGCCCTGGGACCAAGGTGGATATCAAAC.

In particular embodiments, mAb PI3-A12 has a CDRH1 including GDSVKSDDFY (SEQ ID NO: 29); a CDRH2 including IYYGGRT (SEQ ID NO: 30); a CDRH3 including VRVEGLLWFGELFDY (SEQ ID NO: 31); a CDRL1 including NSNIGNNF (SEQ ID NO: 32); a CDRL2 including KDY; and a CDRL3 including AAWQDGLSGPL (SEQ ID NO: 33).

In particular embodiments, mAb PI3-A12 has a heavy chain sequence including QVQLQESGPGLVKPSETLSLTCTVSGDSVKSDDFYWSWIRQPPGKGLEWIGFIYYGGRTYYNP SLSGRGTISVDTSKNHFFLELTSVTAADTAVYYCVRVEGLLWFGELFDYWGQGTLVTVSS (SEQ ID NO: 174) and a light chain sequence including

(SEQ ID NO: 175)
QSVLTQPPSASGTPGQRVTISCSGSNSNIGNNFVYWYQQVPGSAPKVVI
YKDYQRPSGVPDRFSASKSGTSASLTISGLRSDDEAHYYCAAWQDGLSG
PLFGGGTKLTVL.

In particular embodiments, the PI3-A12 antibody includes a variable heavy chain sequence encoded by: CAGGTGCAGCTGCAGGAGTCGGGCCCAGGACTGGTGAAGCCTTCAGAGACCCTGTCCCT CACTTGCACTGTCTCTGGTGACTCCGTCAAGAGTGATGATTTCTACTGGAGTTGGATCCGG CAGCCCCCAGGGAAGGGCCTGGAGTGGATTGGCTTCATCTATTACGGTGGCAGAACTTAC TACAACCCGTCCCTCAGTGGTCGAGGAACCATTTCAGTGGACACGTCCAAGAACCACTTCT TCCTGGAGCTGACCTCTGTGACTGCCGCAGACACGGCCGTATACTACTGTGTCAGGGTCG AAGGATTACTGTGGTTCGGGGAGTTATTCGACTACTGGGGCCAGGGAACCCTGGTCACCG TCTCCTCAG (SEQ ID NO: 34) and a variable lambda light chain sequence encoded by:

(SEQ ID NO: 35)
CAGTCTGTGCTGACGCAGCCACCCTCAGCGTCTGGGACCCCCGGGCAGA
GGGTCACCATCTCTTGTTCTGGAAGCAACTCCAACATCGGAAATAATTT
TGTCTACTGGTACCAACAAGTCCCAGGATCGGCCCCCAAAGTCGTCATT
TACAAGGATTATCAGCGCCCCTCAGGGGTCCCTGACCGATTCTCTGCCT
CCAAGTCTGGCACCTCAGCCTCCCTGACCATCAGCGGGCTCCGGTCCGA
CGATGAGGCCCATTATTACTGTGCAGCATGGCAGGACGGTCTGAGTGGG
CCGTTATTTGGCGGAGGGACCAAGCTGACCGTCCTAG.

In particular embodiments, mAb 3×1 has a CDRH1 including GFTFSSFG (SEQ ID NO: 36); a CDRH2 including ISHSAGFL (SEQ ID NO: 37); a CDRH3 including AKRLAGLPDLEWLLYPNFLDH (SEQ ID NO: 38); a CDRL1 including ILRTYY (SEQ ID NO: 39); a CDRL2 including GKN; and a CDRL3 including SSRDRSGNHVL (SEQ ID NO: 40).

In particular embodiments, mAb 3×1 has a heavy chain sequence including EVQLLESGGGLVQPGGSLRLSCAASGFTFSSFGMSWVRQSPGKGLEWVADISHSAGFLNYAD SVKGRFTVSRDNSKSTLHLQMKSLRAEDTAVYYCAKRLAGLPDLEWLLYPNFLDHWGQGTLV TVSS (SEQ ID NO: 176) and a light chain sequence including

(SEQ ID NO: 177)
SSELTQDPAVSVALGQTVRITCQGDILRTYYVSWYQQKPGQAPLLVIYG
KNNRPSVIPDRFSGSTSGDTASLTITGAQAEDEAEYYCSSRDRSGNHVL
FGGGTKLTVL.

In particular embodiments, the 3×1 xnAb antibody includes a variable heavy chain sequence encoded by: GAGGTGCAGCTGTTGGAGTCTGGGGGAGGCTTGGTACAGCCTGGTGGGTCCCTGAGACT GTCTTGTGCGGCCTCAGGATTCACCTTTAGCAGCTTTGGCATGAGCTGGGTCCGCCAATCT CCAGGAAAGGGGCTGGAGTGGGTCGCAGATATAAGCCATAGTGCTGGCTTCTTAAACTAC GCAGACTCCGTGAAGGGCCGGTTCACCGTCTCCAGAGACAATTCTAAGAGCACGCTGCAT CTCCAAATGAAGAGCCTGAGAGCCGAGGACACGGCCGTATATTACTGTGCGAAAAGACTT GCCGGATTACCAGATTTGGAGTGGTTACTTTACCCTAACTTCTTAGACCACTGGGGCCAGG GAACCCTGGTCACCGTCTCCTCAG (SEQ ID NO: 41) and a variable lambda light chain sequence encoded by:

(SEQ ID NO: 42)
TCTTCTGAGCTGACTCAGGACCCTGCTGTGTCTGTGGCCTTGGGACAGA
CAGTCAGGATCACATGCCAAGGAGACATACTGAGAACCTATTATGTAAG
CTGGTACCAGCAGAAACCAGGACAGGCCCCGCTACTTGTCATCTATGGT
AAAAACAACCGACCCTCAGTGATCCCAGACCGATTCTCTGGCTCCACCT
CAGGAGACACAGCTTCCTTGACCATCACTGGGGCTCAGGCGGAAGATGA
GGCTGAGTATTATTGTAGCTCTCGGGACAGGAGTGGAAACCATGTGCTA
TTCGGCGGAGGGACCAAGCTGACCGTCCTAG.

In particular embodiments, mAb M×R-B11 (also referred to as M×R-01) has a CDRH1 including GFPFSSYK (SEQ ID NO: 43); a CDRH2 including ISASGSYI (SEQ ID NO: 44); a CDRH3 including ARDGGRELSPFEK (SEQ ID NO: 45); a CDRL1 including NSNIGTGYD (SEQ ID NO: 46); a CDRL2 including DNN; and a CDRL3 including QSYDKSLGGWV (SEQ ID NO: 47).

In particular embodiments, M×R-B11 (M×R-01) includes a variable heavy chain having the sequence as set forth in EVQVVESGGGLVKPGGSLRLSCAASGFPFSSYKMDWVRQAPGKGLEWVSSISASGSYINYAD SVKGRFTISRDNAKNSLYLQMKSLRADDTAVYFCARDGGRELSPFEKWGQGILVTVSS (SEQ ID NO: 178) and a variable light chain having the sequence as set forth in

(SEQ ID NO: 179)
QSVLTQPPSVSGAPGQRVTISCTGTNSNIGTGYDVHWYQQLPGTAPKVV
LFDNNNRPSGVPDRFSGSKSGTSAALAITGLQAEDEAVYYCQSYDKSLG
GWVFGGGTKLTVL.

In particular embodiments, the M×R-B11 antibody includes a variable heavy chain encoded by the sequence: GAGGTGCAGGTGGTGGAGTCTGGGGGAGGCCTGGTCAAGCCTGGGGGGTCCCTGAGAC TCTCCTGTGCAGCCTCTGGATTCCCCTTCAGTTCTTATAAGATGGACTGGGTCCGCCAGGC TCCAGGGAAGGGGCTGGAGTGGGTCTCGTCCATCAGTGCTAGTGGAAGTTACATAAACTA TGCAGACTCAGTGAAGGGCCGATTCACCATCTCCAGAGACAACGCCAAGAACTCACTGTAT CTGCAAATGAAAAGCCTGAGAGCCGACGACACGGCTGTATATTTTTGTGCGAGAGACGGC GGAAGAGAACTGAGCCCCTTTGAAAAGTGGGGCCAGGGAATCCTGGTCACCGTCTCCTCA G (SEQ ID NO: 48) and a variable light chain encoded by the sequence:

(SEQ ID NO: 49)
CAGTCTGTGCTGACGCAGCCGCCCTCAGTGTCTGGGGCCCCAGGACAGA
GGGTCACCATCTCCTGCACTGGGACCAACTCCAACATCGGGACAGGTTA
TGATGTACACTGGTACCAGCAGCTTCCGGGAACAGCCCCCAAAGTCGTC
CTCTTTGATAACAACAATCGGCCCTCAGGGGTCCCTGACCGATTCTCTG
GCTCCAAGTCTGGCACTTCAGCCGCCCTGGCCATCACTGGCCTCCAGGC
TGAGGATGAGGCTGTTTATTACTGCCAGTCCTATGACAAGAGCCTGGGT
GGTTGGGTGTTCGGCGGAGGGACCAAGCTGACCGTCCTAG.

In particular embodiments, mAb M×R-D10 (also referred to as M×R-02) has a CDRH1 including GFIFSNYD (SEQ ID NO: 50); a CDRH2 including ITGGSSFI (SEQ ID NO: 51); a CDRH3 including ARDGGRQLSPCEH (SEQ ID NO: 52); a CDRL1 including SSNIGAGYD (SEQ ID NO: 53); a CDRL2 including DNN; and a CDRL3 including QSYDRGLSGWA (SEQ ID NO: 54).

In particular embodiments, mAb M×R-D10 (M×R-02) includes a variable heavy chain having the sequence EVQVVESGGGLVKPGGSLRLSCTASGFIFSNYDMNWVRQAPGKGLEWVASITGGSSFINYAD SVKGRFTISRDNAKKSLYLQMNSLRAEDTAVYYCARDGGRQLSPCEHWGQGTLVTVSS (SEQ ID NO: 180) and a variable light chain having the sequence

(SEQ ID NO: 181)
QSVLTQPPSVSGSPGQRVTISCTGGSSNIGAGYDVHWYQQLPGSAPKLL
MYDSNNRPSGVPDRFSGSKSGTSASLAITGLQAEDEADYYCQSYDRGLS
GWAFGGGTKLTVL.

In particular embodiments, the M×R-D10 antibody includes a variable heavy chain encoded by the sequence: GAGGTGCAGGTGGTGGAGTCGGGGGGAGGCCTGGTCAAGCCTGGGGGGTCCCTGAGAC TCTCCTGTACAGCCTCTGGATTCATATTCAGTAATTATGACATGAACTGGGTCCGCCAGGC TCCAGGGAAGGGCCTGGAGTGGGTCGCCTCCATTACTGGTGGTAGTAGTTTCATAAATTAC GCAGACTCAGTGAAGGGCCGATTCACCATCTCCAGAGACAACGCCAAGAAGTCACTGTAT CTGCAAATGAACAGCCTCAGAGCCGAGGACACGGCTGTCTATTACTGTGCGAGAGATGGC GGGAGACAGTTGAGTCCGTGTGAACATTGGGGCCAGGGAACCCTGGTCACCGTCTCCTCA G (SEQ ID NO: 55) and a variable light chain encoded by the sequence:

(SEQ ID NO: 56)
CAGTCTGTGCTGACGCAGCCGCCCTCAGTGTCTGGGTCCCCAGGGCAGA
GGGTCACCATCTCCTGCACTGGGGGCAGCTCCAACATCGGGGCAGGTTA
TGATGTACACTGGTACCAACAACTTCCAGGATCAGCCCCCAAACTCCTC
ATGTATGATAGTAATAATCGACCCTCAGGGGTCCCTGACCGATTCTCTG
GCTCCAAGTCTGGCACCTCAGCCTCCCTGGCCATCACTGGGCTCCAGGC
TGAGGATGAGGCTGATTATTACTGCCAGTCCTATGACAGGGGCCTGAGT
GGGTGGGCGTTCGGCGGAGGGACCAAGCTGACCGTCCTGG.

Variants of antibodies are also included. Variants of antibodies can include those having one or more conservative amino acid substitutions or one or more non-conservative substitutions that do not adversely affect the binding of the protein.

In particular embodiments, a conservative amino acid substitution may not substantially change the structural characteristics of the reference sequence (e.g., a replacement amino acid should not tend to break a helix that occurs in the reference sequence or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991).

In particular embodiments, a V_{L} region can be derived from or based on a disclosed V_{L} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the disclosed V_{L}. An insertion, deletion or substitution may be anywhere in the V_{L} region, including at the amino- or carboxy-terminus or both ends of this region, provided that each CDR includes zero changes or at most one, two, or three changes and provided an antibody including the modified V_{L} region can still specifically bind its target epitope with an affinity similar to the wild type binding fragment.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} disclosed herein. An insertion, deletion or substitution may be anywhere in the V_{H} region, including at the amino- or carboxy-terminus or both ends of this region, provided that each CDR includes zero changes or at most one, two, or three changes and provided an antibody including the modified V_{H} region can still specifically bind its target epitope with an affinity similar to the wild type binding fragment.

In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to an antibody sequence disclosed herein. In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to a light chain variable region (V_{L}) and/or to a heavy chain variable region (V_{H}), or both, wherein each CDR includes zero changes or at most one, two, or three changes, from the reference antibody disclosed herein or fragment or derivative thereof that specifically binds to the target viral epitope.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions.

As disclosed herein, Fc variants disclosed herein include M252Y/S254T/T256E (YTE) in combination with M428L/N434S (LS) (YTE+LS), YTE+LS in combination with the S239D/A330L/I332E DLE modification set (YTE+LS+DLE), YTE+LS in combination with the G236A/A330L/I332E modification set (YTE+LS+ALE), YTE+LS in combination with the G236A/S239D/A330L modification set (YTE+LS+DAL), and YTE+LS in combination with the G236A/S239D/A330L/I332E modification set (YTE+LS+DALE), and each of these modification sets individually with glycosylation modifications (e.g., M5). These modifications provide administration benefits. As used herein, administration benefits can be one or more of (1) extended half-live, (2) altered binding affinities, (3) altered binding affinity for forming protein complexes, (4) reduced susceptibility to oxidation, (5) reduced immunogenicity; and/or (6) reduced susceptibility to proteolysis.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87:614, 2004; Kanda et al., Biotechnol. Bioeng., 94 (4): 680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence.

Antibodies are generally glycosylated at N297 with a complex glycan that includes fucose. mAbs produced in GnTI-293 cells have an Fc region glycosylated with five mannoses and no fucose. This is the M5 modification described herein. For additional information regarding the M5 modification, see Kanda et al., Glycobiology. 2007 January; 17 (1): 104-118; and Subedi et al., J Vis Exp. 2015 Dec. 28; (106): e53568.

An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. mAbs produced in the presence of fucosylation inhibitor 2-Deoxy-2-fluoro-L-fucose are afucosylated.

Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252:3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, antibodies disclosed herein are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/l of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39 (21) 2011. In some instances, purification by chromatography may not be needed due to the purity of manufacture according the methods described herein.

(ii) Compositions. Any of the antibodies described herein in any exemplary format can be formulated alone or in combination into compositions for administration to subjects. Salts and/or pro-drugs of the antibodies can also be used.

A pharmaceutically acceptable salt includes any salt that retains the activity of the antibody and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

In particular embodiments, the compositions include antibodies of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, a-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. Hank's solution refers to an isotonic buffer solution including inorganic salts and a carbohydrate. Ringer's solution includes sodium chloride, potassium chloride, calcium chloride at physiologic concentrations with sodium bicarbonate (or sodium lactate) to balance pH. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use. Antibodies with the (YTE+LS) modification set are particularly suitable for formulation as an injectable composition.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch. Antibodies with the ALE, DALE, or (YTE+LS+DAL) modification sets and/or glycosylation modifications (e.g., M5 or YTE+LS+M5) are particularly suitable for formulation as an inhalable composition.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly (lactide), poly (glycolide), poly (caprolactone) and poly (lactide)-co (glycolide) (PLG) of desirable lactide: glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl) methyl cellulose (HPMC), HPMC acetate, cellulose acetate, pluronics (e.g., F68/F127), polysorbates, Span® (Croda Americas, Wilmington, Delaware), poly (vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and compositions are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

(iii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of an infection's development, progression, and/or resolution.

A “prophylactic treatment” includes a treatment administered to a subject who does not display signs or symptoms of an infection or displays only early signs or symptoms of an infection such that treatment is administered for the purpose of diminishing or decreasing the risk of developing the infection further. Thus, a prophylactic treatment functions as a preventative treatment against an infection. In particular embodiments, prophylactic treatments reduce, delay, or prevent the worsening of an infection. Antibodies with the (YTE+LS) modification set are particularly suitable to provide prophylactic treatments.

A “therapeutic treatment” includes a treatment administered to a subject who displays symptoms or signs of an infection and is administered to the subject for the purpose of diminishing or eliminating those signs or symptoms of the infection. The therapeutic treatment can reduce, control, or eliminate the presence or activity of the infection and/or reduce control or eliminate side effects of the infection. Antibodies with the ALE, DALE, or (YTE+LS+DAL) modification sets and/or glycosylation modifications (e.g., M5 or YTE+LS+M5) are particularly suitable to provide therapeutic treatments.

Function as an effective amount, prophylactic treatment or therapeutic treatment are not mutually exclusive, and in particular embodiments, administered dosages may accomplish more than one treatment type.

In particular embodiments, therapeutically effective amounts provide anti-infection effects. Anti-infection effects include a reducing or preventing a virus from infecting a cell, decreasing the number of infected cells, decreasing the volume of infected tissue, increasing lifespan, increasing life expectancy, reducing or eliminating infection-associated symptoms (e.g., reduced lung capacity). In particular embodiments, therapeutically effective amounts induce an immune response. The immune response can be against a virus.

Particular embodiments include identifying a subject with a respiratory virus and administering an antibody with a (YTE+LS+DLE), (YTE+LS+DAL), or (YTE+LS+DALE) modification set. Particular embodiments include identifying a subject with a respiratory virus or a flu and administering an antibody with a (YTE+LS+DAL) modification set. Particular embodiments include identifying a subject with a flu and administering an antibody with a (YTE+LS+ALE) or DAL modification set.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of infection, stage of infection, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

As indicated, particular methods include uses in susceptible individuals, such as those undergoing HCT, lung transplant recipients, premature infants, adults over age 65, or those with other health-related issues that increase susceptibility to infection with respiratory viruses.

The pharmaceutical compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration. Antibodies with the (YTE+LS) modification set are particularly suitable for administration by IM or IV injection. Antibodies with the ALE, (DALE), or (YTE+LS+DAL) modification sets and/or glycosylation modifications (e.g., M5 or YTE+LS+M5) are particularly suitable for administration by inhalation.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (IV) EXEMPLARY EMBODIMENTS

1. An antibody with a mutated Fc region including modifications M252Y, S254T, T256E (YTE) and M428L, and N434S (LS).
2. The antibody of embodiment 1, wherein the Fc region further includes modifications S239D, A330L, and I332E (DLE); G236A, A330L, and I332E (ALE); G236A, S239D, A330L (DAL), or G236A, S239D, A330L, and I332E (DALE).
3. The antibody of embodiment 1 or 2, further including a glycosylation modification in the Fc region.
4. The antibody of embodiment 3, wherein the glycosylation modification provides five mannose in the Fc region.
5. The antibody of embodiment 3 or 4, wherein the glycosylation modification results in the absence of fucose in the Fc region.
6. The antibody of embodiment 3, wherein the glycosylation modification provides five mannose in the Fc region and no fucose in the Fc region.
7. The antibody of embodiment 3, wherein the glycosylation modification provides aglycolysation in the Fc region.
8. The antibody of embodiment any of embodiments 1-7, wherein the antibody is an IgG antibody.
9. The antibody of any of embodiments 1-8, wherein the antibody does not include any modifications beyond a recited combination.
10. The antibody of any of embodiments 1-9, wherein the antibody does not include an N434H modification.
11. The antibody of any of embodiments 1-4, wherein the antibody does not include a C2205 modification.
12. The antibody of any of embodiments 1-11, wherein the antibody does not include an N434H modification and does not include a C2205 modification.
13. The antibody of any of embodiments 1-12, including the complementarity determining regions (CDRs) of PI3-E12, PI3-A3, PI3-B5, PI3-A10, PI3-A12, 3×1, M×R-B11, or M×R-D10.
14. The antibody of embodiment 13, wherein the CDRs are according to Kabat, Chothia, Martin, Contact, IMGT, AHo, or North numbering.
15. The antibody of any of embodiments 1-14, having
   a CDRH1 including GFTFSDHY (SEQ ID NO: 1); a CDRH2 including ISSSGSNT (SEQ ID NO: 2); a CDRH3 including ARAKWGTMGRGAPPTIYDH (SEQ ID NO: 3); a CDRL1 including QSLLQSNGNNY (SEQ ID NO: 4); a CDRL2 including LGS; and a CDRL3 including MQALQTPLT (SEQ ID NO: 5);
   a CDRH1 including GFTFSNYW (SEQ ID NO: 8); a CDRH2 including VKEEGSEK (SEQ ID NO: 9); a CDRH3 including AGEVKSGWFGRYFDS (SEQ ID NO: 10); a CDRL1 including QSVGSW (SEQ ID NO: 11); a CDRL2 including KTS; and a CDRL3 including QQYSSFPYT (SEQ ID NO: 12);
   a CDRH1 including GYNFTNYW (SEQ ID NO: 15); a CDRH2 including IYPADSDT (SEQ ID NO: 16); a CDRH3 including ARPSTRWFVPGGMDV (SEQ ID NO: 17); a CDRL1 including QSIGAW (SEQ ID NO: 18); a CDRL2 including KAS; and a CDRL3 including QQHSSYPST (SEQ ID NO: 19);
   a CDRH1 including GFNFNNYG (SEQ ID NO: 22); a CDRH2 including VSFDGSNR (SEQ ID NO: 23); a CDRH3 including SKSKYSDFWSEI (SEQ ID NO: 24); a CDRL1 including QNVMRY (SEQ ID NO: 25); a CDRL2 including DAS; and a CDRL3 including QQRTNHRFS (SEQ ID NO: 26);
   a CDRH1 including GDSVKSDDFY (SEQ ID NO: 29); a CDRH2 including IYYGGRT (SEQ ID NO: 30); a CDRH3 including VRVEGLLWFGELFDY (SEQ ID NO: 31); a CDRL1 including NSNIGNNF (SEQ ID NO: 32); a CDRL2 including KDY; and a CDRL3 including AAWQDGLSGPL (SEQ ID NO: 33);
   a CDRH1 including GFTFSSFG (SEQ ID NO: 36); a CDRH2 including ISHSAGFL (SEQ ID NO: 37); a CDRH3 including AKRLAGLPDLEWLLYPNFLDH (SEQ ID NO: 38); a CDRL1 including ILRTYY (SEQ ID NO: 39); a CDRL2 including GKN; and a CDRL3 including SSRDRSGNHVL (SEQ ID NO: 40);
   a CDRH1 including GFPFSSYK (SEQ ID NO: 43); a CDRH2 including ISASGSYI (SEQ ID NO: 44); a CDRH3 including ARDGGRELSPFEK (SEQ ID NO: 45); a CDRL1 including NSNIGTGYD (SEQ ID NO: 46); a CDRL2 including DNN; and a CDRL3 including QSYDKSLGGWV (SEQ ID NO: 47); or
   a CDRH1 including GFIFSNYD (SEQ ID NO: 50); a CDRH2 including ITGGSSFI (SEQ ID NO: 51); a CDRH3 including ARDGGRQLSPCEH (SEQ ID NO: 52); a CDRL1 including SSNIGAGYD (SEQ ID NO: 53); a CDRL2 including DNN; and a CDRL3 including QSYDRGLSGWA (SEQ ID NO: 54).
16. An antibody of any of embodiments 1-15 including
   a variable heavy chain having a sequence with at least 90% or at least 95% sequence identity to the sequence as set forth in SEQ ID NO: 166 and a variable light chain having a sequence with at least 90% or at least 95% sequence identity to the sequence as set forth in SEQ ID NO: 167;
   a variable heavy chain having a sequence with at least 90% or at least 95% sequence identity to the sequence as set forth in SEQ ID NO: 168 and a variable light chain having a sequence with at least 90% or at least 95% sequence identity to the sequence as set forth in SEQ ID NO: 169;
   a variable heavy chain having a sequence with at least 90% or at least 95% sequence identity to the sequence as set forth in SEQ ID NO: 170 and a variable light chain having a sequence with at least 90% or at least 95% sequence identity to the sequence as set forth in SEQ ID NO: 171;
   a variable heavy chain having a sequence with at least 90% or at least 95% sequence identity to the sequence as set forth in SEQ ID NO: 172 and a variable light chain having a sequence with at least 90% or at least 95% sequence identity to the sequence as set forth in SEQ ID NO: 173;
   a variable heavy chain having a sequence with at least 90% or at least 95% sequence identity to the sequence as set forth in SEQ ID NO: 174 and a variable light chain having a sequence with at least 90% or at least 95% sequence identity to the sequence as set forth in SEQ ID NO: 175;
   a variable heavy chain having a sequence with at least 90% or at least 95% sequence identity to the sequence as set forth in SEQ ID NO: 176 and a variable light chain having a sequence with at least 90% or at least 95% sequence identity to the sequence as set forth in SEQ ID NO: 177;
   a variable heavy chain having a sequence with at least 90% or at least 95% sequence identity to the sequence as set forth in SEQ ID NO: 178 and a variable light chain having a sequence with at least 90% or at least 95% sequence identity to the sequence as set forth in SEQ ID NO: 179; or
   a variable heavy chain having a sequence with at least 90% or at least 95% sequence identity to the sequence as set forth in SEQ ID NO: 180 and a variable light chain having a sequence with at least 90% or at least 95% sequence identity to the sequence as set forth in SEQ ID NO: 181.
17. The antibody of any of embodiments 1-15 having
   a variable heavy chain including the sequence as set forth in SEQ ID NO: 166 and a variable light chain including the sequence as set forth in SEQ ID NO: 167;
   a variable heavy chain including the sequence as set forth in SEQ ID NO: 168 and a variable light chain including the sequence as set forth in SEQ ID NO: 169;
   a variable heavy chain including the sequence as set forth in SEQ ID NO: 170 and a variable light chain including the sequence as set forth in SEQ ID NO: 171;
   a variable heavy chain including the sequence as set forth in SEQ ID NO: 172 and a variable light chain including the sequence as set forth in SEQ ID NO: 173;
   a variable heavy chain including the sequence as set forth in SEQ ID NO: 174 and a variable light chain including the sequence as set forth in SEQ ID NO: 175;
   a variable heavy chain including the sequence as set forth in SEQ ID NO: 176 and a variable light chain including the sequence as set forth in SEQ ID NO: 177;
   a variable heavy chain including the sequence as set forth in SEQ ID NO: 178 and a variable light chain including the sequence as set forth in SEQ ID NO: 179; or
   a variable heavy chain including the sequence as set forth in SEQ ID NO: 180 and a variable light chain including the sequence as set forth in SEQ ID NO: 181
18. The antibody of any of embodiments 1-15 encoded by a sequence having at least 90% or at least 95% sequence identity to the sequence as set forth in SEQ ID NO: 6 and SEQ ID NO: 7;
   at least 90% or at least 95% sequence identity to the sequence as set forth in SEQ ID NO: 13 and SEQ ID NO: 14;
   at least 90% or at least 95% sequence identity to the sequence as set forth in SEQ ID NO: 27 and SEQ ID NO: 28;
   at least 90% or at least 95% sequence identity to the sequence as set forth in SEQ ID NO: 34 and SEQ ID NO: 35;
   at least 90% or at least 95% sequence identity to the sequence as set forth in SEQ ID NO: 41 and SEQ ID NO: 42;
   at least 90% or at least 95% sequence identity to the sequence as set forth in SEQ ID NO: 48 and SEQ ID NO: 49; or
   at least 90% or at least 95% sequence identity to the sequence as set forth in SEQ ID NO: 55 and SEQ ID NO: 56.
19. A composition including an antibody of any of embodiments 1-18 and a pharmaceutically acceptable carrier.
20. The composition of embodiment 19, wherein the pharmaceutically-acceptable carrier includes an aqueous solution.
21. The composition of embodiment 19 or 20, wherein the pharmaceutically-acceptable carrier includes physiological saline.
22. The composition of any of embodiments 19-21, wherein the pharmaceutically-acceptable carrier includes sodium chloride, potassium chloride and calcium chloride.
23. The composition of any of embodiments 19-22, wherein the pharmaceutically-acceptable carrier includes sodium bicarbonate or sodium lactate.
24. The composition of any of embodiments 19-23, wherein the pharmaceutically-acceptable carrier includes inorganic salts.
25. The composition of any of embodiments 19-24, wherein the pharmaceutically-acceptable carrier includes a carbohydrate.
26. The composition of any of embodiments 19-25, wherein the pharmaceutically-acceptable carrier includes an antioxidant, a buffering agent, a chelating agent, an isotonic agent, a preservative, and/or a stabilizer.
27. The composition of any of embodiments 19-26, wherein the pharmaceutically-acceptable carrier includes a release modifier.
28. The composition of any of embodiments 19-27, wherein the composition includes a therapeutically-effective amount of the antibody for administration to a subject.
29. The composition of embodiment 19, wherein the composition is formulated for intramuscular or intravenous administration and includes an antibody with a (YTE+LS) modification set.
30. The composition of embodiment 19, wherein the composition is formulated for inhalation and includes an antibody with an ALE, DALE, M5, (YTE+LS+DAL), or (YTE+LS+M5) modification set.
31. A method of providing an anti-viral effect in a subject in need thereof including administering a therapeutically effective amount of the composition of any of embodiments 19-30 to the subject thereby providing the anti-viral effect.
32. The method of embodiment 31, wherein the anti-viral effect includes an anti-HPIV3 effect, an anti-HPIV1 effect, an anti-respiratory syncytial virus (RSV) effect, an anti-human metapneumovirus (HMPV) effect, an anti-SARS-CoV-2 effect, and/or an anti-influenza (flu) effect.
33. The method of any of embodiment 31 or 32, wherein the subject in need thereof is immunocompromised.
34. The method of any of embodiments 31-33, wherein the subject in need thereof is a hematopoietic stem cell transplant (HCT) recipient, a lung transplant recipient, a premature infant, a person over 65 years of age, a homeless person, or a person with lung disease.
35. The method of any of embodiments 31-34, wherein the subject in need thereof has chronic obstructive pulmonary disease (COPD).
36. The method of embodiment 31, wherein the subject has a respiratory virus and the composition includes an antibody with a (YTE+LS+DLE), (YTE+LS+DAL), (YTE+LS+DALE), (YTE+LS+ALE), M5, or (YTE+LS+M5) modification set.
37. The method of embodiment 31, wherein the respiratory virus includes HPIV3, HPIV1, RSV, HMPV, or SARS-CoV-2.
38. The method of embodiment 31, wherein the subject has a respiratory virus or a flu and the composition includes an antibody with a (YTE+LS+DAL) or (YTE+LS+ALE) modification set.
39. The method of embodiment 31, wherein the subject has a a flu and the composition includes an antibody with a (YTE+LS+ALE) or DAL modification set.
40. The method of embodiment 31, wherein the therapeutically effective amount provides a prophylactic anti-viral effect.
41. The method of embodiment 41, wherein the administered composition includes a (YTE+LS) modification set.
42. The method of embodiment 40 or 41, wherein the administering is through injection.
43. The method of embodiment 42, wherein the injection is intramuscular (IM) or intravenous (IV) injection.
44. The method of embodiment 31, wherein the therapeutically effective amount provides a therapeutic anti-viral effect.
45. The method of embodiment 44, wherein the administered composition includes an ALE, DALE, (YTE+LS+DAL), M5, or (YTE+LS+M5) modification set.
46. The method of embodiment 39 or 40, wherein the administering is through direct administration to the respiratory tract.
47. The method of embodiment 39 or 40, wherein the administering is through inhalation.

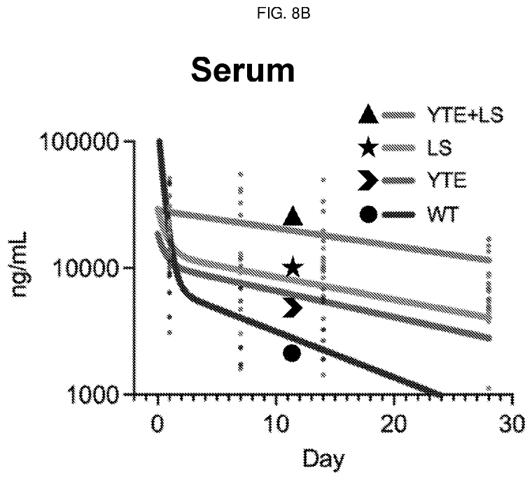

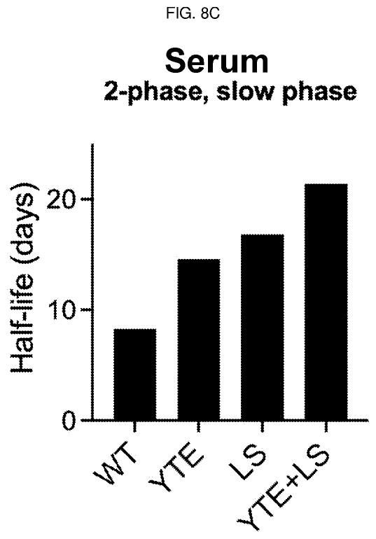

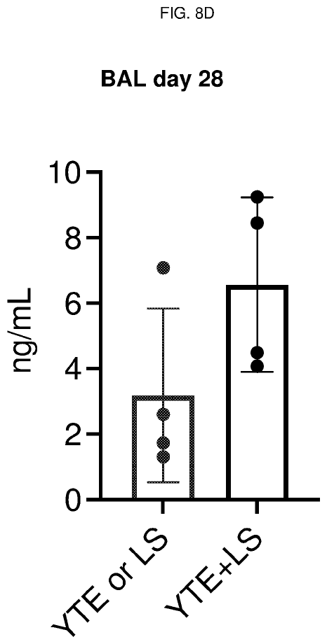

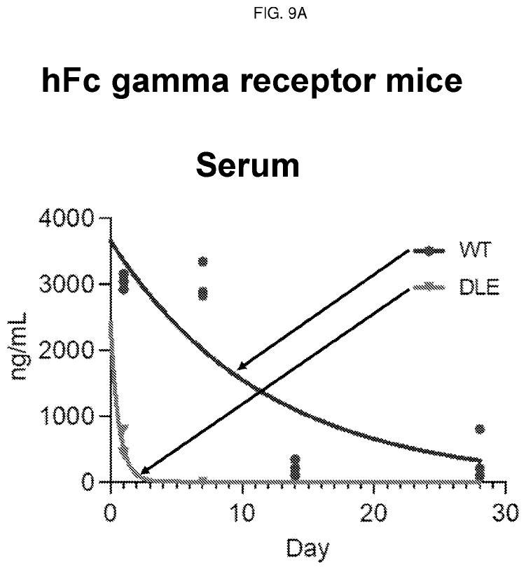

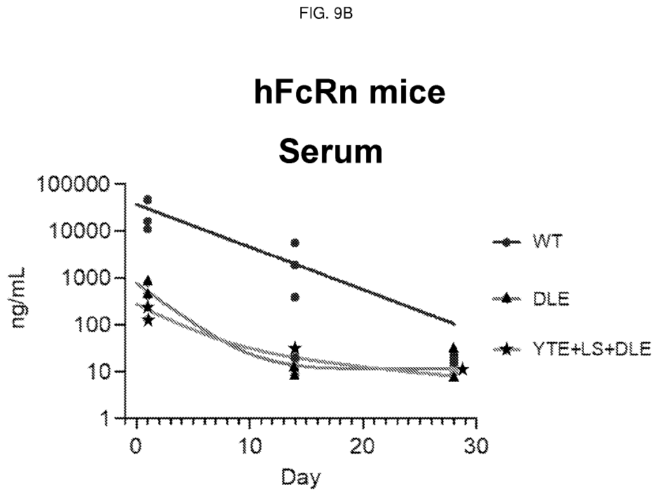

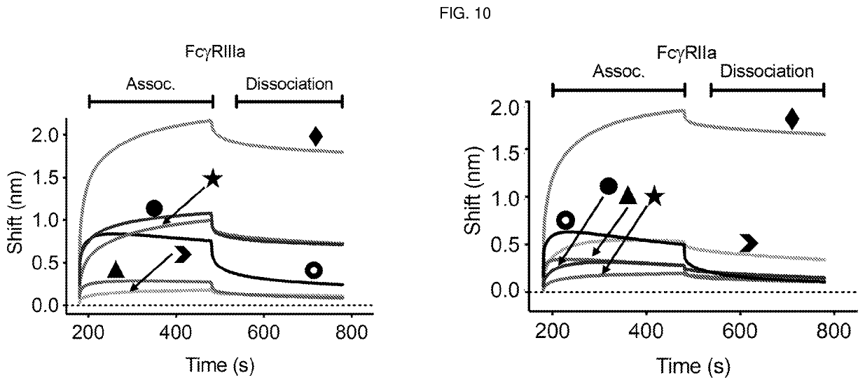

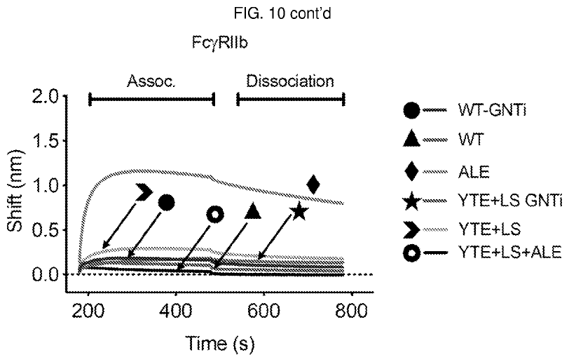

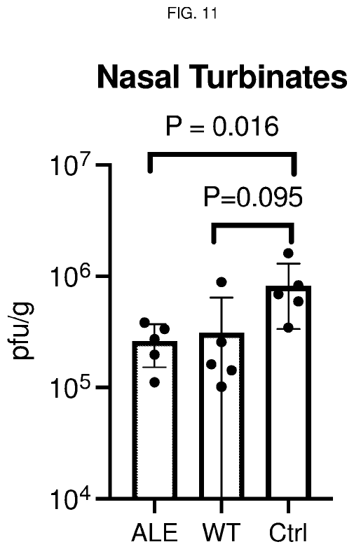

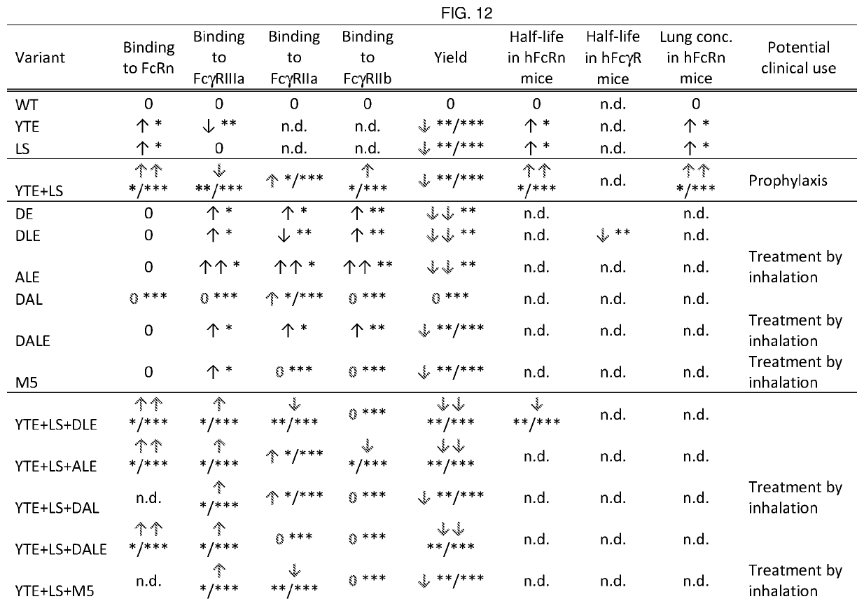

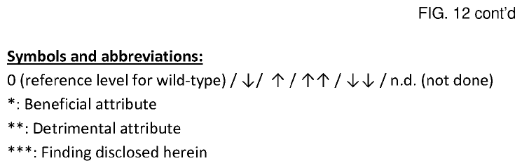

(v) Example 1. Methods: To confirm that described in vitro results translate into prolonged half-life and increased respiratory bioavailability in vivo, 5 mg/kg M×R, M×R^{YTE}, M×R^{LS}, or M×R^{YTE+LS} was administered intravenously to 6-8-week-old, humanized FcRn transgenic mice (hFcRn Tg32). Serum was collected longitudinally over 28 days (FIG. 8A). BAL was performed at the end of study and on a separate cohort of mice at week one to measure respiratory bioavailability. Levels of mAb in serum and BAL were measured by ELISA. Compartmental and non-compartmental PK parameters were analyzed using descriptive statistics. Half-life was calculated using a two-phase exponential decay model. In addition, Cₘₐₓ, Tₘₐₓ, and area under the curve will be calculated. For statistical analysis and reproducibility, each group consisted of at least four mice, and each experiment was repeated independently at least twice.

Combining the YTE and LS Fc modifications extended the half-life and respiratory bioavailability of IgG1 to a greater extent than either modification alone. Increased concentrations of YTE+LS-modified M×R in BAL fluid was also observed.
Prophetic Example 1. Similar to FcRn, inter-species differences between binding of IgG with FcγRIIIa can manifest as discrepancies between results from animal models and clinical trials in humans. Even NHP models have limitations, because NHPs uniformly express the high affinity V176 allele of FcγRIIIa, whereas the majority of humans express the low affinity F176 allele. Crowley et al., Front Immunol 10, 697, (2019). However, humanized FcγRIIIa transgenic mice can model Fc effector functions and predict efficacy of human IgG. Yamin et al., Nature 599, 465-470, (2021); Smith et al., Proc Natl Acad Sci USA 109, 6181-6186, (2012). Therefore, these mice will be used to further confirm the therapeutic efficacy of disclosed Fc variants of M×R against infection with RSV and HMPV.

To confirm that improved FcγRIIIa binding in vitro translates into improved therapeutic efficacy in vivo, 16-18-week-old, humanized FcγRIIIa transgenic mice will be infected intranasally with RSV or HMPV. Older mice will be used since they are more susceptible to RSV infection, similar to humans. Graham et al., J Med Virol 26, 153-162, (1988). One day later, M×R, M×R^{YTE}, M×R^{DLE}, M×R^{YTE+LS+DLE}, or PBS control will be injected intravenously. Mice will be weighed daily, and lungs collected on day 5 for histopathology and to measure viral replication by quantitative PCR and plaque assay. Blood will also be collected to compare cell surface binding of the Fc variants by flow cytometry. For statistical analysis and reproducibility, each group will consist of at least five mice, and each experiment will be repeated independently at least twice.

The boost in FcγRIIIa binding conferred by the DLE modification set will lead to greater suppression of viral replication.
Prophetic Example 2. mAbs produced in the presence of fucosylation inhibitor 2-Deoxy-2-fluoro-L-fucose are afucosylated. These antibodies increase binding to FcgRIIIa and antibody dependent cellular cytotoxicity with no increased clearance by the liver (see PMID 23493549, 17012310, and 26779721).

Afucosylated M×R^{WT} and M×R^{YTE+LS} will be produced in 293 cells in the presence of fucosylation inhibitor 2-Deoxy-2-fluoro-L-fucose. Binding to Fcγ receptors, clearance in humanized FcRn mice, and efficacy as treatment in humanized FcgR mice will be tested.

(vi) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (IIe), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, IIe, Phe, Met, and Trp; Group 11 (aliphatic): Gly, Ala, Val, Leu, and IIe; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: IIe (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); IIe (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or modifications that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding fragment (of, for example, a binding fragment) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding fragments may be classified as “high affinity” or “low affinity”. In particular embodiments, “high affinity” binding fragments refer to those binding fragments with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding fragments refer to those binding fragments with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding fragment may have “enhanced affinity,” which refers to a selected or engineered binding fragments with stronger binding to a cognate binding molecule than a wild type (or parent) binding fragment. For example, enhanced affinity may be due to a Ka (equilibrium association constant) for the cognate binding molecule that is higher than the reference binding fragment or due to a Kd (dissociation constant) for the cognate binding molecule that is less than that of the reference binding fragment, or due to an off-rate (Koff) for the cognate binding molecule that is less than that of the reference binding fragment. A variety of assays are known for detecting binding fragments that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in increased FcRn binding, lung bioavailability, and half-life following administration, as disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An antibody with a mutated Fc region including modifications M252Y, S254T, T256E (YTE) and modifications M428L and N434S (LS), wherein said antibody optionally further includes modifications
S239D, A330L, and I332E (DLE);
G236A, A330L, and I332E (ALE);
G236A, S239D, A330L (DAL), or
G236A, S239D, A330L, and I332E (DALE).

2. The antibody of claim 1, further comprising a glycosylation modification in the Fc region.

3. The antibody of claim 2, wherein the glycosylation modification provides five mannose in the Fc region.

4. The antibody of claim 2, wherein the glycosylation modification results in the absence of fucose in the Fc region.

5. The antibody of claim 2, wherein the glycosylation modification provides five mannose in the Fc region and no fucose in the Fc region.

6. The antibody of claim 2, wherein the glycosylation modification provides aglycolysation in the Fc region.

7. The antibody of claim 1, wherein the antibody is an IgG antibody.

8. The antibody of claim 1, wherein the antibody does not comprise any modifications beyond a recited combination.

9. The antibody of claim 1, wherein the antibody does not include an N434H modification.

10. The antibody of claim 1, wherein the antibody does not include a C2205 modification.

11. The antibody of claim 1, wherein the antibody does not include an N434H modification and does not include a C2205 modification.

12. The antibody of claim 1, having a six-member complementarity determining region (CDR) set of PI3-E12, PI3-A3, PI3-B5, PI3-A10, PI3-A12, 3×1, M×R-B11, or M×R-D10.

13. The antibody of claim 12, wherein the CDRs are according to Kabat, Chothia, Martin, Contact, IMGT, AHo, or North numbering.

14. The antibody of claim 1, having
a CDRH1 having GFTFSDHY (SEQ ID NO: 1), a CDRH2 having ISSSGSNT (SEQ ID NO: 2), a CDRH3 having ARAKWGTMGRGAPPTIYDH (SEQ ID NO: 3), a CDRL1 having QSLLOSNGNNY (SEQ ID NO: 4), a CDRL2 having LGS, and a CDRL3 having MQALQTPLT (SEQ ID NO: 5),
a CDRH1 having GFTFSNYW (SEQ ID NO: 8), a CDRH2 having VKEEGSEK (SEQ ID NO: 9), a CDRH3 having AGEVKSGWFGRYFDS (SEQ ID NO: 10), a CDRL1 having QSVGSW (SEQ ID NO: 11), a CDRL2 having KTS, and a CDRL3 having QQYSSFPYT (SEQ ID NO: 12);
a CDRH1 having GYNFTNYW (SEQ ID NO: 15), a CDRH2 having IYPADSDT (SEQ ID NO: 16), a CDRH3 having ARPSTRWFVPGGMDV (SEQ ID NO: 17), a CDRL1 having QSIGAW (SEQ ID NO: 18), a CDRL2 having KAS, and a CDRL3 having QQHSSYPST (SEQ ID NO: 19);
a CDRH1 having GFNFNNYG (SEQ ID NO: 22), a CDRH2 having VSFDGSNR (SEQ ID NO: 23), a CDRH3 having SKSKYSDFWSEI (SEQ ID NO: 24), a CDRL1 having QNVMRY (SEQ ID NO: 25), a CDRL2 having DAS, and a CDRL3 having QQRTNHRFS (SEQ ID NO: 26);
a CDRH1 having GDSVKSDDFY (SEQ ID NO: 29), a CDRH2 having IYYGGRT (SEQ ID NO: 30), a CDRH3 having VRVEGLLWFGELFDY (SEQ ID NO: 31), a CDRL1 having NSNIGNNF (SEQ ID NO: 32), a CDRL2 having KDY, and a CDRL3 having AAWQDGLSGPL (SEQ ID NO: 33);
a CDRH1 having GFTFSSFG (SEQ ID NO: 36), a CDRH2 having ISHSAGFL (SEQ ID NO: 37), a CDRH3 having AKRLAGLPDLEWLLYPNFLDH (SEQ ID NO: 38), a CDRL1 having ILRTYY (SEQ ID NO: 39), a CDRL2 having GKN, and a CDRL3 having SSRDRSGNHVL (SEQ ID NO: 40);
a CDRH1 having GFPFSSYK (SEQ ID NO: 43), a CDRH2 having ISASGSYI (SEQ ID NO: 44), a CDRH3 having ARDGGRELSPFEK (SEQ ID NO: 45), a CDRL1 having NSNIGTGYD (SEQ ID NO: 46), a CDRL2 having DNN, and a CDRL3 having QSYDKSLGGWV (SEQ ID NO: 47); or
a CDRH1 having GFIFSNYD (SEQ ID NO: 50), a CDRH2 having ITGGSSFI (SEQ ID NO: 51), a CDRH3 having ARDGGRQLSPCEH (SEQ ID NO: 52), a CDRL1 having SSNIGAGYD (SEQ ID NO: 53), a CDRL2 having DNN, and a CDRL3 having QSYDRGLSGWA (SEQ ID NO: 54).

15. The antibody of claim 1 having
a variable heavy chain having a sequence with at least 95% sequence identity to the sequence as set forth in SEQ ID NO: 166 and a variable light chain having a sequence with r at least 95% sequence identity to the sequence as set forth in SEQ ID NO: 167;
a variable heavy chain having a sequence with at least 95% sequence identity to the sequence as set forth in SEQ ID NO: 168 and a variable light chain having a sequence with at least 95% sequence identity to the sequence as set forth in SEQ ID NO: 169;
a variable heavy chain having a sequence with at least 95% sequence identity to the sequence as set forth in SEQ ID NO: 170 and a variable light chain having a sequence with at least 95% sequence identity to the sequence as set forth in SEQ ID NO: 171;
a variable heavy chain having a sequence with at least 95% sequence identity to the sequence as set forth in SEQ ID NO: 172 and a variable light chain having a sequence with at least 95% sequence identity to the sequence as set forth in SEQ ID NO: 173;
a variable heavy chain having a sequence with at least 95% sequence identity to the sequence as set forth in SEQ ID NO: 174 and a variable light chain having a sequence with or at least 95% sequence identity to the sequence as set forth in SEQ ID NO: 175;
a variable heavy chain having a sequence with at least 95% sequence identity to the sequence as set forth in SEQ ID NO: 176 and a variable light chain having a sequence with at least 95% sequence identity to the sequence as set forth in SEQ ID NO: 177;
a variable heavy chain having a sequence with at least 95% sequence identity to the sequence as set forth in SEQ ID NO: 178 and a variable light chain having a sequence with at least 95% sequence identity to the sequence as set forth in SEQ ID NO: 179; or
a variable heavy chain having a sequence with at least 95% sequence identity to the sequence as set forth in SEQ ID NO: 180 and a variable light chain having a sequence with at least 95% sequence identity to the sequence as set forth in SEQ ID NO: 181.

16. The antibody of claim 1 having
a variable heavy chain having the sequence as set forth in SEQ ID NO: 166 and a variable light chain having the sequence as set forth in SEQ ID NO: 167;
a variable heavy chain having the sequence as set forth in SEQ ID NO: 168 and a variable light chain having the sequence as set forth in SEQ ID NO: 169;
a variable heavy chain having the sequence as set forth in SEQ ID NO: 170 and a variable light chain having the sequence as set forth in SEQ ID NO: 171;
a variable heavy chain having the sequence as set forth in SEQ ID NO: 172 and a variable light chain having the sequence as set forth in SEQ ID NO: 173;
a variable heavy chain having the sequence as set forth in SEQ ID NO: 174 and a variable light chain having the sequence as set forth in SEQ ID NO: 175;
a variable heavy chain having the sequence as set forth in SEQ ID NO: 176 and a variable light chain having the sequence as set forth in SEQ ID NO: 177;
a variable heavy chain having the sequence as set forth in SEQ ID NO: 178 and a variable light chain having the sequence as set forth in SEQ ID NO: 179; or
a variable heavy chain having the sequence as set forth in SEQ ID NO: 180 and a variable light chain having the sequence as set forth in SEQ ID NO: 181.

17. The antibody of claim 1 encoded by a sequence having
at least 95% sequence identity to the sequence as set forth in SEQ ID NO: 6 and SEQ ID NO: 7;
at least 95% sequence identity to the sequence as set forth in SEQ ID NO: 13 and SEQ ID NO: 14;
at least 95% sequence identity to the sequence as set forth in SEQ ID NO: 27 and SEQ ID NO: 28;
at least 95% sequence identity to the sequence as set forth in SEQ ID NO: 34 and SEQ ID NO: 35;
at least 95% sequence identity to the sequence as set forth in SEQ ID NO: 41 and SEQ ID NO: 42;
at least 95% sequence identity to the sequence as set forth in SEQ ID NO: 48 and SEQ ID NO: 49; or
at least 95% sequence identity to the sequence as set forth in SEQ ID NO: 55 and SEQ ID NO: 56.

18. A composition having the antibody of claim 1 and a pharmaceutically-acceptable carrier.

19. The composition of claim 18, wherein the pharmaceutically-acceptable carrier comprises an aqueous solution.

20. The composition of claim 18, wherein the pharmaceutically-acceptable carrier comprises physiological saline.

21. The composition of claim 18, wherein the pharmaceutically-acceptable carrier comprises sodium chloride, potassium chloride and calcium chloride.

22. The composition of claim 18, wherein the pharmaceutically-acceptable carrier comprises sodium bicarbonate or sodium lactate.

23. The composition of claim 18, wherein the pharmaceutically-acceptable carrier comprises inorganic salts.

24. The composition of claim 18, wherein the pharmaceutically-acceptable carrier comprises a carbohydrate.

25. The composition of claim 18, wherein the pharmaceutically-acceptable carrier comprises an antioxidant, a buffering agent, a chelating agent, an isotonic agent, a preservative, and/or a stabilizer.

26. The composition of claim 18, wherein the pharmaceutically-acceptable carrier comprises a release modifier.

27. The composition of claim 18, wherein the composition comprises a therapeutically-effective amount of the antibody for administration to a subject.

28. The composition of claim 18, wherein the composition is formulated for intramuscular or intravenous administration and includes an antibody with a (YTE+LS) modification set.

29. The composition of claim 18, wherein the composition is formulated for inhalation and includes an antibody with an ALE, DALE, (YTE+LS+DAL), M5 or (YTE+LS+M5) modification set.

30. A method of providing an anti-viral effect in a subject in need thereof having administering a therapeutically effective amount of the composition of claim 18 to the subject thereby providing the anti-viral effect.

31. The method of claim 30, wherein the anti-viral effect comprises an anti-HPIV3 effect, an anti-HPIV1 effect, an anti-respiratory syncytial virus (RSV) effect, an anti-human metapneumovirus (HMPV) effect, an anti-SARS-CoV-2 effect, and/or an anti-influenza (flu) effect.

32. The method of claim 31, wherein the subject in need thereof is immunocompromised.

33. The method of claim 31, wherein the subject in need thereof is a hematopoietic stem cell transplant (HCT) recipient, a lung transplant recipient, a premature infant, a person over 65 years of age, a homeless person, or a person with lung disease.

34. The method of claim 31, wherein the subject has chronic obstructive pulmonary disease (COPD).

35. The method of claim 31, wherein the subject has a respiratory virus and the composition includes an antibody with a (YTE+LS+DLE), (YTE+LS+DAL), (YTE+LS+ALE), (YTE+LS+DALE), M5 or (YTE+LS+M5) modification set.

36. The method of claim 35, wherein the respiratory virus includes HPIV3, HPIV1, RSV, HMPV, or SARS-CoV-2.

37. The method of claim 30, wherein the subject has a respiratory virus or a flu and the composition includes an antibody with a (YTE+LS+ALE), (YTE+LS+DAL), M5 or (YTE+LS+M5) modification set.

38. The method of claim 30, wherein the subject has a flu and the composition includes an antibody with a (YTE+LS+ALE) or DAL modification set.

39. The method of claim 30, wherein the therapeutically effective amount provides a prophylactic anti-viral effect.

40. The method of claim 30, wherein the administered composition includes a (YTE+LS) modification set.

41. The method of claim 40, wherein the administering is through injection.

42. The method of embodiment 41, wherein the injection is intramuscular (IM) or intravenous (IV) injection.

43. The method of claim 30, wherein the therapeutically effective amount provides a therapeutic anti-viral effect.

44. The method of claim 43, wherein the administered composition includes an ALE, DALE, (YTE+LS+DAL), M5 or (YTE+LS+M5) modification set.

45. The method of claim 44, wherein the administering is through direct administration to the respiratory tract.

46. The method of claim 44, wherein the administering is through inhalation.


SEQUENCE LISTING (181 sequences)

SEQ ID NO: 1 - protein, 8 aa, synthetic construct
  source 1..8: note = CDRH1 region of mAB PI3-E12
GFTFSDHY

SEQ ID NO: 2 - protein, 8 aa, synthetic construct
  source 1..8: note = CDRH2 region of mAb PI3-E12
ISSSGSNT

SEQ ID NO: 3 - protein, 19 aa, synthetic construct
  source 1..19: note = CDRH3 region of mAb PI3-E12
ARAKWGTMGRGAPPTIYDH

SEQ ID NO: 4 - protein, 11 aa, synthetic construct
  source 1..11: note = CDRL1 region of mAb PI3-E12
QSLLQSNGNNY

SEQ ID NO: 5 - protein, 9 aa, synthetic construct
  source 1..9: note = CDRL3 region of mAb PI3-E12
MQALQTPLT

SEQ ID NO: 6 - DNA, 378 bp, synthetic construct
  source 1..378: mol_type = other DNA; note = variable heavy chain region of PI3-E12
caggtgcagctgttggagtctgggggaaagttggtcaagcctggagggtccctgagactctcctgtgcagcctctggattcaccttcagtgaccactacatgatctggatccgccaggctcccgggaaggggctggagtggatttcatacataagtagtagtggtagtaacacaatctacgcagactctttgatgggccgattcaccatctccagagacaacgccaagaactctctgtatctacaaatgaacagcctgaggaccgaggacacggccgtttattactgtgcgagagcaaagtggggtactatgggtcggggagcacccccgacaatttatgaccactggggccagggaaccctggtcaccgtctcctca

SEQ ID NO: 7 - DNA, 337 bp, synthetic construct
  source 1..337: mol_type = other DNA; note = variable kappa light chain of PI3-E12
gatattgtgatgactcagtctccactctccctgcccgtcacccctggagagccggcctccatctcctgtaggtctagtcagagcctcctgcaaagtaatggaaacaattatttggagtggtacctgcagaagccagggcagtctccacaactcctgatctatttgggttccaatcgggcctccggggtccctgacaggttcagtggcagtggatcaggcacagattttacactgaagatcagcagagtggaggctgaggatgctggggtttattactgcatgcaagctctacaaactccgctcactttcggcggagggaccaaggtggagatcaaac

SEQ ID NO: 8 - protein, 8 aa, synthetic construct
  source 1..8: note = CDRH1 region of mAb PI3-A3
GFTFSNYW

SEQ ID NO: 9 - protein, 8 aa, synthetic construct
  source 1..8: note = CDRH2 region of mAb PI3-A3
VKEEGSEK

SEQ ID NO: 10 - protein, 15 aa, synthetic construct
  source 1..15: note = CDRH3 region of mAb PI3-A3
AGEVKSGWFGRYFDS

SEQ ID NO: 11 - protein, 6 aa, synthetic construct
  source 1..6: note = CDRL1 region of mAb PI3-A3
QSVGSW

SEQ ID NO: 12 - protein, 9 aa, synthetic construct
  source 1..9: note = CDRL3 region of mAb PI3-A3
QQYSSFPYT

SEQ ID NO: 13 - protein, 367 aa, synthetic construct
  source 1..367: note = variable heavy chain sequence of PI3-A3
GAGGTGCAGCTGGTGGAGTCTGGGGGAGGCTTGGTCCAGCCTGGGGGGTCCCTGAGACTCTCCTGTACAGCCTCTGGATTCACCTTTAGCAATTATTGGATGAGCTGGGTCCGCCAGGCTCCAGGGAAGGGGCTGGAGTGGGTGGCCAATGTGAAGGAAGAAGGAAGTGAGAAACACTATGTAGACTCTGTGAAGGGCCGATTCACCATCTCCAGAGACAACGCCAAGAACTCAGTGTATCTGCAGATGAGCAGCCTGAGAGCCGAGGACACGGCTGTCTATTACTGTGCGGGAGAGGTTAAGAGTGGCTGGTTCGGTCGGTACTTTGACTCCTGGGGCCAGGGAACCCTGGTCACCGTCTCCTCAG

SEQ ID NO: 14 - DNA, 322 bp, synthetic construct
  source 1..322: mol_type = other DNA; note = variable kappa light chain region of PI3-A3
gacatccagatgacccagtctccttccaccctgtctgcatctgttggagacagagtcaccatcaattgccgggccagtcagagtgttggtagctggttggcctggtatcagcagaaaccagggaaagcccctaagctcctgatgtataagacatctactttacaaagaggggtcccatcaaggttcagcggcagtggatctgggacagaattcactctcaccatcagcagcctgcagcctgatgattttgcagcttattactgccaacagtatagtagttttccgtacacttttggccaggggaccaagctggagatcaaac

SEQ ID NO: 15 - protein, 8 aa, synthetic construct
  source 1..8: note = CDRH1 region of mAb PI3-B5
GYNFTNYW

SEQ ID NO: 16 - protein, 8 aa, synthetic construct
  source 1..8: note = CDRH2 region of mAb PI3-B5
IYPADSDT

SEQ ID NO: 17 - protein, 15 aa, synthetic construct
  source 1..15: note = CDRH3 region of mAb PI3-B5
ARPSTRWFVPGGMDV

SEQ ID NO: 18 - protein, 6 aa, synthetic construct
  source 1..6: note = CDRL1 region of mAb PI3-B5
QSIGAW

SEQ ID NO: 19 - protein, 9 aa, synthetic construct
  source 1..9: note = CDRL3 region of mAb PI3-B5
QQHSSYPST

SEQ ID NO: 20 - DNA, 366 bp, synthetic construct
  source 1..366: mol_type = other DNA; note = Variable heavy chain region of PI3-B5
gaggtgcagctggtgcagtctggagcagaggtgaaaaagcccggggagtctctgaggatctcctgtaagggttctggatacaactttaccaactactggatcgcctgggtgcgccagatgcccgggaaaggcctggagtggatggggatcatctatcctgctgactcggataccagatacagcccgtccttccaaggccaagtcaccatctcagccgacaagtccatcaccaccgcctacctgcagtggagcagcctgaaggcctcggacaccgccatatactactgtgcgagaccgagtactaggtggttcgtccctggcggtatggacgtctggggccaaggcaccacggtcatcgtctcctca

SEQ ID NO: 21 - DNA, 322 bp, synthetic construct
  source 1..322: mol_type = other DNA; note = Variable kappa light chain region of PI3-B5
gacatccagatgacccagtctccttccaccctgtctgcttctgtaggagacagagtcaccatttcttgccgggccactcagagtattggtgcctggttggcctggtatcagcagaaaccaggggaaccccctaagctcctgatctataaggcgtctactttagagagtggggtcccatcaaggttcagcggcagtggatctgggacagaattcactctcaccatcagcagcctgcagcctgatgattctgcaacttattactgccaacagcatagtagttatccttctactttcggcggagggaccaaggtggagatcaaac

SEQ ID NO: 22 - protein, 8 aa, synthetic construct
  source 1..8: note = CDRH1 region of mAb PI3-A10
GFNFNNYG

SEQ ID NO: 23 - protein, 8 aa, synthetic construct
  source 1..8: note = CDRH2 region of mAb PI3-A10
VSFDGSNR

SEQ ID NO: 24 - protein, 12 aa, synthetic construct
  source 1..12: note = CDRH3 region of mAb PI3-A10
SKSKYSDFWSEI

SEQ ID NO: 25 - protein, 6 aa, synthetic construct
  source 1..6: note = CDRL1 region of mAb PI3-A10
QNVMRY

SEQ ID NO: 26 - protein, 9 aa, synthetic construct
  source 1..9: note = CDRL3 region of mAb PI3-A10
QQRTNHRFS

SEQ ID NO: 27 - DNA, 358 bp, synthetic construct
  source 1..358: mol_type = other DNA; note = variable heavy chain region of PI3-A10
caggtgcagctggtggagtctgggggaggcgtggtccggcctgggaggtccctgagactctcctgtgtagcctctggattcaacttcaataactatgggctgcagtggatccgccaggctccaggcaaggggctggagtgggtggcaggtgtctcgtttgatgggagtaatagatattatgcagactccgtgaagggccgagtcaccatatccagagacgattccaagaacaccctgtatctagaaatgaacagcctgagagctgaggacacaggaatatattactgttcgaagtccaagtactccgacttttggagcgaaatatggggccagggaaccctggtcaccgtctcctcag

SEQ ID NO: 28 - DNA, 322 bp, synthetic construct
  source 1..322: mol_type = other DNA; note = variable kappa light chain region of PI3-A10
gaaattgtgttgacacagtctccagccaccctgtctttgtctccaggggaaagagccaccctctcctgcagggccagtcagaatgttatgaggtacttagcctggtaccaacagagacctggccaggctcccagactcctcttctatgatgcatccagccgggccactggcatcccagcccggttcactgccagtgggtctgggacagacttcactctcaccatcagcggcctcgagcctggagattttgcagtttattactgtcagcagcgtaccaaccatagattctctttcggccctgggaccaaggtggatatcaaac

SEQ ID NO: 29 - protein, 10 aa, synthetic construct
  source 1..10: note = CDRH1 region of mAb PI3-A12
GDSVKSDDFY

SEQ ID NO: 30 - protein, 7 aa, synthetic construct
  source 1..7: note = CDRH2 region of mAb PI3-A12
IYYGGRT

SEQ ID NO: 31 - protein, 15 aa, synthetic construct
  source 1..15: note = CDRH3 region of mAb PI3-A12
VRVEGLLWFGELFDY

SEQ ID NO: 32 - protein, 8 aa, synthetic construct
  source 1..8: note = CDRL1 region of mAb PI3-A12
NSNIGNNF

SEQ ID NO: 33 - protein, 11 aa, synthetic construct
  source 1..11: note = CDRL3 region of mAb PI3-A12
AAWQDGLSGPL

SEQ ID NO: 34 - DNA, 370 bp, synthetic construct
  source 1..370: mol_type = other DNA; note = variable heavy chain region of PI3-A12
caggtgcagctgcaggagtcgggcccaggactggtgaagccttcagagaccctgtccctcacttgcactgtctctggtgactccgtcaagagtgatgatttctactggagttggatccggcagcccccagggaagggcctggagtggattggcttcatctattacggtggcagaacttactacaacccgtccctcagtggtcgaggaaccatttcagtggacacgtccaagaaccacttcttcctggagctgacctctgtgactgccgcagacacggccgtatactactgtgtcagggtcgaaggattactgtggttcggggagttattcgactactggggccagggaaccctggtcaccgtctcctcag

SEQ ID NO: 35 - DNA, 331 bp, synthetic construct
  source 1..331: mol_type = other DNA; note = variable lambda light chain region of PI3-A12
cagtctgtgctgacgcagccaccctcagcgtctgggacccccgggcagagggtcaccatctcttgttctggaagcaactccaacatcggaaataattttgtctactggtaccaacaagtcccaggatcggcccccaaagtcgtcatttacaaggattatcagcgcccctcaggggtccctgaccgattctctgcctccaagtctggcacctcagcctccctgaccatcagcgggctccggtccgacgatgaggcccattattactgtgcagcatggcaggacggtctgagtgggccgttatttggcggagggaccaagctgaccgtcctag

SEQ ID NO: 36 - protein, 8 aa, synthetic construct
  source 1..8: note = CDRH1 region of mAb 3x1
GFTFSSFG

SEQ ID NO: 37 - protein, 8 aa, synthetic construct
  source 1..8: note = CDRH2 region of mAb 3x1
ISHSAGFL

SEQ ID NO: 38 - protein, 21 aa, synthetic construct
  source 1..21: note = CDRH3 region of mAb 3x1
AKRLAGLPDLEWLLYPNFLDH

SEQ ID NO: 39 - protein, 6 aa, synthetic construct
  source 1..6: note = CDRL1 region of CDRH1
ILRTYY

SEQ ID NO: 40 - protein, 11 aa, synthetic construct
  source 1..11: note = CDRL3 region of mAb 3x1
SSRDRSGNHVL

SEQ ID NO: 41 - DNA, 385 bp, synthetic construct
  source 1..385: mol_type = other DNA; note = variable heavy chain region of 3x1 xnAb
gaggtgcagctgttggagtctgggggaggcttggtacagcctggtgggtccctgagactgtcttgtgcggcctcaggattcacctttagcagctttggcatgagctgggtccgccaatctccaggaaaggggctggagtgggtcgcagatataagccatagtgctggcttcttaaactacgcagactccgtgaagggccggttcaccgtctccagagacaattctaagagcacgctgcatctccaaatgaagagcctgagagccgaggacacggccgtatattactgtgcgaaaagacttgccggattaccagatttggagtggttactttaccctaacttcttagaccactggggccagggaaccctggtcaccgtctcctcag

SEQ ID NO: 42 - DNA, 325 bp, synthetic construct
  source 1..325: mol_type = other DNA; note = variable lambda light chain region of 3x1 xnAb
tcttctgagctgactcaggaccctgctgtgtctgtggccttgggacagacagtcaggatcacatgccaaggagacatactgagaacctattatgtaagctggtaccagcagaaaccaggacaggccccgctacttgtcatctatggtaaaaacaaccgaccctcagtgatcccagaccgattctctggctccacctcaggagacacagcttccttgaccatcactggggctcaggcggaagatgaggctgagtattattgtagctctcgggacaggagtggaaaccatgtgctattcggcggagggaccaagctgaccgtcctag

SEQ ID NO: 43 - protein, 8 aa, synthetic construct
  source 1..8: note = CDRH1 region of mAb MxR-B11
GFPFSSYK

SEQ ID NO: 44 - protein, 8 aa, synthetic construct
  source 1..8: note = CDRH2 region of mAb MxR-B11
ISASGSYI

SEQ ID NO: 45 - protein, 13 aa, synthetic construct
  source 1..13: note = CDRH3 region of mAb MxR-B11
ARDGGRELSPFEK

SEQ ID NO: 46 - protein, 9 aa, synthetic construct
  source 1..9: note = CDRL1 region of mAb MxR-B11
NSNIGTGYD

SEQ ID NO: 47 - protein, 11 aa, synthetic construct
  source 1..11: note = CDRL3 region of mAb MxR-B11
QSYDKSLGGWV

SEQ ID NO: 48 - DNA, 361 bp, synthetic construct
  source 1..361: mol_type = other DNA; note = MxR-B11 antibody variable heavy chain sequence
gaggtgcaggtggtggagtctgggggaggcctggtcaagcctggggggtccctgagactctcctgtgcagcctctggattccccttcagttcttataagatggactgggtccgccaggctccagggaaggggctggagtgggtctcgtccatcagtgctagtggaagttacataaactatgcagactcagtgaagggccgattcaccatctccagagacaacgccaagaactcactgtatctgcaaatgaaaagcctgagagccgacgacacggctgtatatttttgtgcgagagacggcggaagagaactgagcccctttgaaaagtggggccagggaatcctggtcaccgtctcctcag

SEQ ID NO: 49 - DNA, 334 bp, synthetic construct
  source 1..334: mol_type = other DNA; note = MxR-B11 antibody variable light chain sequence
cagtctgtgctgacgcagccgccctcagtgtctggggccccaggacagagggtcaccatctcctgcactgggaccaactccaacatcgggacaggttatgatgtacactggtaccagcagcttccgggaacagcccccaaagtcgtcctctttgataacaacaatcggccctcaggggtccctgaccgattctctggctccaagtctggcacttcagccgccctggccatcactggcctccaggctgaggatgaggctgtttattactgccagtcctatgacaagagcctgggtggttgggtgttcggcggagggaccaagctgaccgtcctag

SEQ ID NO: 50 - protein, 8 aa, synthetic construct
  source 1..8: note = CDRH1 region of mAb MxR-D10
GFIFSNYD

SEQ ID NO: 51 - protein, 8 aa, synthetic construct
  source 1..8: note = CDRH2 region of mAb MxR-D10
ITGGSSFI

SEQ ID NO: 52 - protein, 13 aa, synthetic construct
  source 1..13: note = CDRH3 region of mAb MxR-D10
ARDGGRQLSPCEH

SEQ ID NO: 53 - protein, 9 aa, synthetic construct
  source 1..9: note = CDRL1 region of mAb MxR-D10
SSNIGAGYD

SEQ ID NO: 54 - protein, 11 aa, synthetic construct
  source 1..11: note = CDRL3 region of mAb MxR-D10
QSYDRGLSGWA

SEQ ID NO: 55 - DNA, 361 bp, synthetic construct
  source 1..361: mol_type = other DNA; note = MxR-D10 antibody variable heavy chain sequence
gaggtgcaggtggtggagtcggggggaggcctggtcaagcctggggggtccctgagactctcctgtacagcctctggattcatattcagtaattatgacatgaactgggtccgccaggctccagggaagggcctggagtgggtcgcctccattactggtggtagtagtttcataaattacgcagactcagtgaagggccgattcaccatctccagagacaacgccaagaagtcactgtatctgcaaatgaacagcctcagagccgaggacacggctgtctattactgtgcgagagatggcgggagacagttgagtccgtgtgaacattggggccagggaaccctggtcaccgtctcctcag

SEQ ID NO: 56 - DNA, 334 bp, synthetic construct
  source 1..334: mol_type = other DNA; note = MxR-D10 antibody variable light chain sequence
cagtctgtgctgacgcagccgccctcagtgtctgggtccccagggcagagggtcaccatctcctgcactgggggcagctccaacatcggggcaggttatgatgtacactggtaccaacaacttccaggatcagcccccaaactcctcatgtatgatagtaataatcgaccctcaggggtccctgaccgattctctggctccaagtctggcacctcagcctccctggccatcactgggctccaggctgaggatgaggctgattattactgccagtcctatgacaggggcctgagtgggtgggcgttcggcggagggaccaagctgaccgtcctgg

SEQ ID NO: 57-165 - skipped (109 entries)

SEQ ID NO: 166 - protein, 126 aa, synthetic construct
  source 1..126: note = PI3-E12 heavy chain sequence
QVQLLESGGKLVKPGGSLRLSCAASGFTFSDHYMIWIRQAPGKGLEWISYISSSGSNTIYADSLMGRFTISRDNAKNSLYLQMNSLRTEDTAVYYCARAKWGTMGRGAPPTIYDHWGQGTLVTVSS

SEQ ID NO: 167 - protein, 112 aa, synthetic construct
  source 1..112: note = PI3-E12 light chain sequence
DIVMTQSPLSLPVTPGEPASISCRSSQSLLQSNGNNYLEWYLQKPGQSPQLLIYLGSNRASGVPDRFSGSGSGTDFTLKISRVEAEDAGVYYCMQALQTPLTFGGGTKVEIK

SEQ ID NO: 168 - protein, 122 aa, synthetic construct
  source 1..122: note = mAb PI3-A3 heavy chain sequence
EVQLVESGGGLVQPGGSLRLSCTASGFTFSNYWMSWVRQAPGKGLEWVANVKEEGSEKHYVDSVKGRFTISRDNAKNSVYLQMSSLRAEDTAVYYCAGEVKSGWFGRYFDSWGQGTLVTVSS

SEQ ID NO: 169 - protein, 107 aa, synthetic construct
  source 1..107: note = mAb PI3-A3 light chain sequence
DIQMTQSPSTLSASVGDRVTINCRASQSVGSWLAWYQQKPGKAPKLLMYKTSTLQRGVPSRFSGSGSGTEFTLTISSLQPDDFAAYYCQQYSSFPYTFGQGTKLEIK

SEQ ID NO: 170 - protein, 122 aa, synthetic construct
  source 1..122: note = mAb PI3-B5 heavy chain sequence
EVQLVQSGAEVKKPGESLRISCKGSGYNFTNYWIAWVRQMPGKGLEWMGIIYPADSDTRYSPSFQGQVTISADKSITTAYLQWSSLKASDTAIYYCARPSTRWFVPGGMDVWGQGTTVIVSS

SEQ ID NO: 171 - protein, 107 aa, synthetic construct
  source 1..107: note = mAb PI3-B5 light chain sequence
DIQMTQSPSTLSASVGDRVTISCRATQSIGAWLAWYQQKPGEPPKLLIYKASTLESGVPSRFSGSGSGTEFTLTISSLQPDDSATYYCQQHSSYPSTFGGGTKVEIK

SEQ ID NO: 172 - protein, 119 aa, synthetic construct
  source 1..119: note = mAb PI3-A10 heavy chain sequence
QVQLVESGGGVVRPGRSLRLSCVASGFNFNNYGLQWIRQAPGKGLEWVAGVSFDGSNRYYADSVKGRVTISRDDSKNTLYLEMNSLRAEDTGIYYCSKSKYSDFWSEIWGQGTLVTVSS

SEQ ID NO: 173 - protein, 107 aa, synthetic construct
  source 1..107: note = mAb PI3-A10 light chain sequence
EIVLTQSPATLSLSPGERATLSCRASQNVMRYLAWYQQRPGQAPRLLFYDASSRATGIPARFTASGSGTDFTLTISGLEPGDFAVYYCQQRTNHRFSFGPGTKVDIK

SEQ ID NO: 174 - protein, 123 aa, synthetic construct
  source 1..123: note = mAb PI3-A12 heavy chain sequence
QVQLQESGPGLVKPSETLSLTCTVSGDSVKSDDFYWSWIRQPPGKGLEWIGFIYYGGRTYYNPSLSGRGTISVDTSKNHFFLELTSVTAADTAVYYCVRVEGLLWFGELFDYWGQGTLVTVSS

SEQ ID NO: 175 - protein, 110 aa, synthetic construct
  source 1..110: note = mAb PI3-A12 light chain sequence
QSVLTQPPSASGTPGQRVTISCSGSNSNIGNNFVYWYQQVPGSAPKVVIYKDYQRPSGVPDRFSASKSGTSASLTISGLRSDDEAHYYCAAWQDGLSGPLFGGGTKLTVL

SEQ ID NO: 176 - protein, 128 aa, synthetic construct
  source 1..128: note = mAb 3x1 heavy chain sequence
EVQLLESGGGLVQPGGSLRLSCAASGFTFSSFGMSWVRQSPGKGLEWVADISHSAGFLNYADSVKGRFTVSRDNSKSTLHLQMKSLRAEDTAVYYCAKRLAGLPDLEWLLYPNFLDHWGQGTLVTVSS

SEQ ID NO: 177 - protein, 108 aa, synthetic construct
  source 1..108: note = mAb 3x1 light chain sequence
SSELTQDPAVSVALGQTVRITCQGDILRTYYVSWYQQKPGQAPLLVIYGKNNRPSVIPDRFSGSTSGDTASLTITGAQAEDEAEYYCSSRDRSGNHVLFGGGTKLTVL

SEQ ID NO: 178 - protein, 120 aa, synthetic construct
  source 1..120: note = MxR-B11 variable heavy chain sequence
EVQVVESGGGLVKPGGSLRLSCAASGFPFSSYKMDWVRQAPGKGLEWVSSISASGSYINYADSVKGRFTISRDNAKNSLYLQMKSLRADDTAVYFCARDGGRELSPFEKWGQGILVTVSS

SEQ ID NO: 179 - protein, 111 aa, synthetic construct
  source 1..111: note = MxR-B11 variable light chain sequence
QSVLTQPPSVSGAPGQRVTISCTGTNSNIGTGYDVHWYQQLPGTAPKVVLFDNNNRPSGVPDRFSGSKSGTSAALAITGLQAEDEAVYYCQSYDKSLGGWVFGGGTKLTVL

SEQ ID NO: 180 - protein, 120 aa, synthetic construct
  source 1..120: note = mAb MxR-D10 variable heavy chain sequence
EVQVVESGGGLVKPGGSLRLSCTASGFIFSNYDMNWVRQAPGKGLEWVASITGGSSFINYADSVKGRFTISRDNAKKSLYLQMNSLRAEDTAVYYCARDGGRQLSPCEHWGQGTLVTVSS

SEQ ID NO: 181 - protein, 111 aa, synthetic construct
  source 1..111: note = mAb MxR-D10 variable light chain sequence
QSVLTQPPSVSGSPGQRVTISCTGGSSNIGAGYDVHWYQQLPGSAPKLLMYDSNNRPSGVPDRFSGSKSGTSASLAITGLQAEDEADYYCQSYDRGLSGWAFGGGTKLTVL